(19) United States
(12) Patent Application Publication
(10) Pub. No.: US 2020/0008709 A1
(43) Pub. Date: Jan. 9, 2020

(54) METHODS AND APPARATUSES FOR TREATMENT OF APNEA BASED ON ULTRASOUND DATA

(71) Applicant: Butterfly Network, Inc., Guilford, CT (US)

(72) Inventors: John Martin Martin, Crownsville, MD (US); Tyler S. Ralston, Clinton, CT (US)

(21) Appl. No.: 16/383,878

(22) Filed: Apr. 15, 2019

Related U.S. Application Data
(60) Provisional application No. 62/695,248, filed on Jul. 9, 2018.

(51) Int. Cl. A61B 5/08; A61B 8/08; A61M 16/00; A61M 16/06

(52) U.S. Cl. CPC A61B 5/0826; A61B 5/0816; A61B 5/087; A61M 16/0003; A61M 16/0666; A61B 8/08

(57) ABSTRACT

Aspects of the technology described herein relate to delivery of pressure based on ultrasound data. Certain aspects relate to receiving ultrasound data collected from a subject by a wearable ultrasound device, determining that the ultrasound data indicates apnea (e.g., absence of lung sliding or movement of internal abdominal organs), and based on determining that the first ultrasound data indicates apnea, increase pressure being delivered to the subject. To increase the pressure, a positive airway pressure device may increase the pressure it generates, an adapter may route power to the positive airway pressure device, or a valve may permit air to flow from the positive airway pressure device to the subject. Increasing pressure may be triggered by an activation signal transmitted by the wearable ultrasound device or a processing device. The wearable ultrasound device may be a patch configured to couple to the subject's skin.

### CROSS-REFERENCE TO RELATED APPLICATIONS

The present application claims the benefit of priority under 35 U.S.C. § 119 to U.S. Provisional Patent Application Ser. No. 62/695,248, titled “METHODS AND APPARATUSES FOR TREATMENT OF APNEA BASED ON ULTRASOUND DATA,” filed on Jul. 9, 2018, which is incorporated by reference herein in its entirety.

### FIELD

Generally, the aspects of the technology described herein relate to treatment of apnea. Some aspects relate to delivery of pressure to a subject's airway based on ultrasound data.

### BACKGROUND

Sleep apnea is a disorder in which a subject's breathing periodically stops or becomes shallower. The disorder may cause tiredness and increase the risk of stroke, cardiovascular disease, and diabetes. Sleep apnea may be treated using positive airway pressure devices, which deliver pressure to a subject's airways in order to maintain the subject's airways open. However, the delivery of pressure may be uncomfortable for the subject. For example, the subject may need to exhale against the pressure being forced into the subject's airways. Additionally, delivery of pressure may not always be necessary, such as when the subject is not experiencing apnea (e.g., lack of breathing). Lack of subject comfort while using positive airway pressure devices may contribute to lack of subject compliance in treating sleep apnea with such devices.

### SUMMARY

According to one aspect, a method includes receiving, with processing circuitry, first ultrasound data collected from a subject by a wearable ultrasound device; automatically determining, by the processing circuitry, that the first ultrasound data indicates apnea; and responsive to a determination by the processing circuitry that the first ultrasound data indicates apnea, automatically controlling, by the processing circuitry, a positive airway pressure device coupled to the subject to increase pressure delivered to an airway of the subject.

In some embodiments, automatically determining that the first ultrasound data indicates apnea comprises automatically determining that the subject is experiencing apnea. In some embodiments, automatically determining that the first ultrasound data indicates apnea comprises inputting the first ultrasound data to a statistical model trained to determine whether inputted ultrasound data indicates apnea. In some embodiments, automatically determining that the first ultrasound data indicates apnea comprises determining that the first ultrasound data indicates an absence of lung sliding. In some embodiments, automatically determining that the first ultrasound data indicates apnea comprises determining that the first ultrasound data indicates an absence of movement of internal abdominal organs. In some embodiments, the method further comprises receiving, with the processing circuitry, second ultrasound data collected from the subject by the wearable ultrasound device; automatically determining, by the processing circuitry, that the second ultrasound data does not indicate apnea; and responsive to a determination by the processing circuitry that the second ultrasound data does not indicate apnea, automatically controlling, by the processing circuitry, the positive airway pressure device to decrease pressure delivered to the airway of the subject. In some embodiments, automatically determining that the second ultrasound data does not indicate apnea comprises automatically determining that the subject is not experiencing apnea. In some embodiments, automatically determining that the second ultrasound data does not indicate apnea comprises inputting the first ultrasound data to a statistical model trained to determine whether inputted ultrasound data does not indicate apnea. In some embodiments, automatically determining that the second ultrasound data does not indicate apnea comprises determining that the second ultrasound data indicates lung sliding. In some embodiments, automatically determining that the second ultrasound data does not indicate apnea comprises determining that the second ultrasound data indicates movement of internal abdominal organs.

In some embodiments, the positive airway pressure device comprises the processing circuitry; and automatically controlling the positive airway pressure device to increase the pressure delivered to the airway of the subject comprises increasing pressure generated by the positive airway pressure device. In some embodiments, the positive airway pressure device comprises the processing circuitry; and automatically controlling the positive airway pressure device to decrease the pressure delivered to the airway of the subject comprises decreasing pressure generated by the positive airway pressure device.

In some embodiments, an adapter coupled between the positive airway pressure device and a power source comprises the processing circuitry; and automatically controlling the positive airway pressure device to increase the pressure delivered to the airway of the subject comprises routing, by the adapter, power from the power source to the positive airway pressure device. In some embodiments, an adapter coupled between the positive airway pressure device and a power source comprises the processing circuitry; and automatically controlling the positive airway pressure device to decrease the pressure delivered to the airway of the subject comprises ceasing to route, by the adapter, power from the power source to the positive airway pressure device.

In some embodiments, a valve coupled between the positive airway pressure device and the subject comprises the processing circuitry; and automatically controlling the positive airway pressure device to increase the pressure delivered to the airway of the subject comprises permitting, by the valve, air to flow from the positive airway pressure device to the airway of the subject. In some embodiments, a valve coupled between the positive airway pressure device and the subject comprises the processing circuitry; and automatically controlling the positive airway pressure device to decrease the pressure delivered to the airway of the subject comprises preventing, by the valve, air to flow from the positive airway pressure device to the airway of the subject.

In some embodiments, automatically controlling the positive airway pressure device to increase the pressure delivered to the airway of the subject comprises generating, by the processing circuitry, an activation signal configured to trigger the positive airway device to increase the pressure generated by the positive airway pressure device. In some embodiments, automatically controlling the positive airway pressure device to increase the pressure delivered to the airway of the subject comprises generating, by the processing circuitry, a deactivation signal configured to trigger the positive airway device to decrease the pressure generated by the positive airway pressure device. In some embodiments, automatically controlling the positive airway pressure device to increase the pressure delivered to the airway of the subject comprises generating, by the processing circuitry, an activation signal configured to trigger an adapter coupled between the positive airway pressure device and a power source to route the power from the power source to the positive airway pressure device. In some embodiments, automatically controlling the positive airway pressure device to increase the pressure delivered to the airway of the subject comprises generating, by the processing circuitry, a deactivation signal configured to trigger an adapter coupled between the positive airway pressure device and a power source to cease to route the power from the power source to the positive airway pressure device. In some embodiments, automatically controlling the positive airway pressure device to increase the pressure delivered to the airway of the subject comprises generating, by the processing circuitry, an activation signal configured to trigger a valve coupled between the positive airway pressure device and the subject to permit the air to flow from the positive airway pressure device to the airway of the subject. In some embodiments, automatically controlling the positive airway pressure device to increase the pressure delivered to the airway of the subject comprises generating, by the processing circuitry, a deactivation signal configured to trigger a valve coupled between the positive airway pressure device and the subject to prevent the air to flow from the positive airway pressure device to the airway of the subject.

In some embodiments, the wearable ultrasound device comprises the processing circuitry. In some embodiments, the wearable ultrasound device comprises a patch configured to couple to the subject's skin. In some embodiments, a processing device in communication with the wearable ultrasound device comprises the processing circuitry. Some aspects include an apparatus including processing circuitry configured to perform the above methods.

According to another aspect, a method includes receiving, by a positive airway pressure device coupled to a subject and configured to generate pressure delivered to an airway of the subject, an activation signal from: a processing device in communication with a wearable ultrasound device coupled to the subject; or the wearable ultrasound device; and based on receiving the activation signal, increasing the pressure generated by the positive airway pressure device. In some embodiments, the method further includes receiving, by the positive airway pressure device, a deactivation signal from: the processing device in communication with the wearable ultrasound device coupled to the subject; or the wearable ultrasound device; and based on receiving the deactivation signal, decreasing the pressure generated by the positive airway pressure device.

According to another aspect, an apparatus comprises a positive airway pressure device coupled to a subject and configured to: generate pressure delivered to an airway of the subject; receive an activation signal from: a processing device in communication with a wearable ultrasound device coupled to the subject; or the wearable ultrasound device; and based on receiving the activation signal, increase the pressure delivered to the airway of the subject. In some embodiments, the positive airway pressure device is further configured to: receive a deactivation signal from: the processing device in communication with the wearable ultrasound device coupled to the subject; or the wearable ultrasound device; and based on receiving the deactivation signal, decrease the pressure delivered to the airway of the subject.

According to another aspect, a method includes receiving, by an adapter coupled between a positive airway pressure device coupled to a subject and a power source, an activation signal from: a processing device in communication with a wearable ultrasound device coupled to the subject; or the wearable ultrasound device; and based on receiving the activation signal, routing power from the power source to the positive airway pressure device. In some embodiments, the method further includes receiving, by the adapter, a deactivation signal from: the processing device in communication with the wearable ultrasound device coupled to the subject; or the wearable ultrasound device; and based on receiving the deactivation signal, ceasing to route power from the power source to the positive airway pressure device.

According to another aspect, an apparatus includes an adapter coupled between a positive airway pressure device coupled to a subject and a power source, wherein the adapter is configured to: receive an activation signal from: a processing device in communication with a wearable ultrasound device coupled to the subject; or the wearable ultrasound device; and based on receiving the activation signal, route power from the power source to the positive airway pressure device. In some embodiments, the adapter is further configured to: receive a deactivation signal from: the processing device in communication with the wearable ultrasound device coupled to the subject; or the wearable ultrasound device; and based on receiving the deactivation signal, prevent routing of power from the power source to the positive airway pressure device.

According to another aspect, a method includes receiving, by a valve coupled between a positive airway pressure device and a subject, an activation signal from: a processing device in communication with a wearable ultrasound device coupled to the subject; or the wearable ultrasound device; and based on receiving the activation signal, permitting air to flow from the positive airway pressure device to the subject. In some embodiments, the method includes receiving, by the valve, a deactivation signal from: the processing device in communication with the wearable ultrasound device coupled to the subject; or the wearable ultrasound device; and based on receiving the deactivation signal, preventing air from flowing from the positive airway pressure device to the subject.

According to another aspect, an apparatus includes a valve coupled between a positive airway pressure device and a subject, wherein the valve is configured to: receive an activation signal from: a processing device in communication with a wearable ultrasound device coupled to the subject; or the wearable ultrasound device; and based on receiving the activation signal, permit air to flow from the positive airway pressure device to the subject. In some embodiments, the valve is further configured to: receive a deactivation signal from: the processing device in communication with the wearable ultrasound device coupled to the subject; or the wearable ultrasound device; and based on receiving the deactivation signal, prevent air from flowing from the power source to the positive airway pressure device.

According to another aspect, an apparatus comprises processing circuitry configured to: receive first ultrasound data collected from a subject by a wearable ultrasound device; automatically determine that the first ultrasound data indicates apnea; and responsive to a determination that the first ultrasound data indicates apnea, automatically control a positive airway pressure device coupled to the subject to increase pressure delivered to an airway of the subject.

In some embodiments, the processing circuitry is configured, when automatically determining that the first ultrasound data indicates apnea, to automatically determine that the subject is experiencing apnea. In some embodiments, the processing circuitry is configured, when automatically determining that the first ultrasound data indicates apnea, to input the first ultrasound data to a statistical model trained to determine whether inputted ultrasound data indicates apnea. In some embodiments, the processing circuitry is configured, when automatically determining that the first ultrasound data indicates apnea, to determine that the first ultrasound data indicates an absence of lung sliding. In some embodiments, the processing circuitry is configured, when automatically determining that the first ultrasound data indicates apnea, to determine that the first ultrasound data indicates an absence of movement of internal abdominal organs.

In some embodiments, the processing circuitry is further configured to: receive second ultrasound data collected from the subject by the wearable ultrasound device; automatically determine that the second ultrasound data does not indicate apnea; and responsive to a determination by the processing circuitry that second ultrasound data does not indicate apnea, automatically control the positive airway pressure device to decrease pressure delivered to the airway of the subject. In some embodiments, the processing circuitry is configured, when automatically determining that the second ultrasound data does not indicate apnea, to automatically determine that the subject is not experiencing apnea. In some embodiments, the processing circuitry is configured, when determining that the second ultrasound data does not indicate apnea, to input the first ultrasound data to a statistical model trained to determine whether inputted ultrasound data does not indicate apnea. In some embodiments, the processing circuitry is configured, when automatically determining that the second ultrasound data does not indicate apnea, to determine that the second ultrasound data indicates lung sliding. In some embodiments, the processing circuitry is configured, when automatically determining that the second ultrasound data does not indicate apnea, to determine that the second ultrasound data indicates movement of internal abdominal organs.

In some embodiments, the positive airway pressure device comprises the processing circuitry; and the processing circuitry is configured, when automatically controlling the positive airway pressure device to increase the pressure delivered to the airway of the subject, to increase pressure generated by the positive airway pressure device. In some embodiments, the positive airway pressure device comprises the processing circuitry; and the processing circuitry is configured, when automatically controlling the positive airway pressure device to decrease the pressure delivered to the airway of the subject, to decrease pressure generated by the positive airway pressure device. In some embodiments, an adapter coupled between the positive airway pressure device and a power source comprises the processing circuitry; and the processing circuitry is configured, when automatically controlling the positive airway pressure device to increase the pressure delivered to the airway of the subject, to route power from the power source to the positive airway pressure device. In some embodiments, an adapter coupled between the positive airway pressure device and a power source comprises the processing circuitry; and the processing circuitry is configured, when automatically controlling the positive airway pressure device to decrease the pressure delivered to the airway of the subject, to cease to route power from the power source to the positive airway pressure device. In some embodiments, a valve coupled between the positive airway pressure device and the subject comprises the processing circuitry; and the processing circuitry is configured, when automatically controlling the positive airway pressure device to increase the pressure delivered to the airway of the subject, to permit air to flow from the positive airway pressure device to the airway of the subject. In some embodiments, a valve coupled between the positive airway pressure device and the subject comprises the processing circuitry; and the processing circuitry is configured, when automatically controlling the positive airway pressure device to decrease the pressure delivered to the airway of the subject, to prevent air to flow from the positive airway pressure device to the airway of the subject.

In some embodiments, the processing circuitry is configured, when automatically controlling the positive airway pressure device to increase the pressure delivered to the airway of the subject, to generate an activation signal configured to trigger the positive airway device to increase the pressure generated by the positive airway pressure device. In some embodiments, the processing circuitry is configured, when automatically controlling the positive airway pressure device to increase the pressure delivered to the airway of the subject, to generate a deactivation signal configured to trigger the positive airway device to decrease the pressure generated by the positive airway pressure device. In some embodiments, the processing circuitry is configured, when automatically controlling the positive airway pressure device to increase the pressure delivered to the airway of the subject, to generate an activation signal configured to trigger an adapter coupled between the positive airway pressure device and a power source to route the power from the power source to the positive airway pressure device. In some embodiments, the processing circuitry is configured, when automatically controlling the positive airway pressure device to increase the pressure delivered to the airway of the subject, to generate a deactivation signal configured to trigger an adapter coupled between the positive airway pressure device and a power source to cease to route the power from the power source to the positive airway pressure device. In some embodiments, the wearable ultrasound device comprises the processing circuitry. In some embodiments, wherein the wearable ultrasound device comprises a patch configured to couple to the subject's skin. In some embodiments, a processing device in communication with the wearable ultrasound device comprises the processing circuitry.

In some embodiments, the processing circuitry is configured, when automatically controlling the positive airway pressure device to increase the pressure delivered to the airway of the subject, to generate an activation signal configured to trigger a valve coupled between the positive airway pressure device and the subject to permit the air to flow from the positive airway pressure device to the airway of the subject. In some embodiments, the processing circuitry is configured, when automatically controlling the positive airway pressure device to increase the pressure delivered to the airway of the subject, to generate a deactivation signal configured to trigger a valve coupled between the positive airway pressure device and the subject to prevent the air to flow from the positive airway pressure device to the airway of the subject.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example system for delivering pressure to a subject based on ultrasound data, where the system includes a wearable ultrasound device, a processing device, and a positive airway pressure device, in accordance with certain embodiments described herein;

FIG. 2 illustrates another example system for delivering pressure to a subject based on ultrasound data, where the system includes a wearable ultrasound device, a processing device, a positive airway pressure device, and an adapter, in accordance with certain embodiments described herein;

FIG. 3 illustrates another example system for delivering pressure to a subject based on ultrasound data, where the system includes a wearable ultrasound device, a processing device, a positive airway pressure device, and a valve, in accordance with certain embodiments described herein;

FIG. 4 is a schematic block diagram of an example system for delivering pressure to a subject based on ultrasound data, where the system includes a wearable ultrasound device, a processing device, and a positive airway pressure device, in accordance with certain embodiments described herein;

FIG. 5 is a schematic block diagram of another example system for delivering pressure to a subject based on ultrasound data, where the system includes a wearable ultrasound device and a positive airway pressure device, in accordance with certain embodiments described herein;

FIG. 6 is a schematic block diagram of another example system for delivering pressure to a subject based on ultrasound data, where the system includes a wearable ultrasound device, a processing device, a positive airway pressure device, and an adapter, in accordance with certain embodiments described herein;

FIG. 7 is a schematic block diagram of another example system for delivering pressure to a subject based on ultrasound data, where the system includes a wearable ultrasound device, a positive airway pressure device, and an adapter, in accordance with certain embodiments described herein;

FIG. 8 is a schematic block diagram of another example system for delivering pressure to a subject based on ultrasound data, where the system includes a wearable ultrasound device, a processing device, a positive airway pressure device, and a valve, in accordance with certain embodiments described herein;

FIG. 9 is a schematic block diagram of another example system for delivering pressure to a subject based on ultrasound data, where the system includes a wearable ultrasound device, a positive airway pressure device, and a valve, in accordance with certain embodiments described herein;

FIG. 10 illustrates an ultrasound patch, in accordance with certain embodiments described herein;

FIG. 11 illustrates the ultrasound patch coupled to a subject, in accordance with certain embodiments described herein;

FIG. 12 shows an exploded view of the ultrasound patch, in accordance with certain embodiments described herein;

FIG. 13 shows another exploded view of the ultrasound patch, in accordance with certain embodiments described herein;

FIG. 14 shows an alternative fastening mechanism for the ultrasound patch, in accordance with certain embodiments described herein;

FIG. 15 shows an example of the ultrasound patch fastened to a subject using the strap of FIG. 14, in accordance with certain embodiments described herein;

FIG. 16 shows an example process for delivering pressure to a subject based on ultrasound data, in accordance with certain embodiments described herein;

FIG. 17 shows another example process for delivering pressure to a subject, in accordance with certain embodiments described herein;

FIG. 18 shows another example process for delivering pressure to a subject, in accordance with certain embodiments described herein;

FIG. 19 shows an example process for delivering pressure to a subject, in accordance with certain embodiments described herein; and

FIG. 20 shows an example convolutional neural network that is configured to analyze an image, in accordance with certain embodiments described herein.

### DETAILED DESCRIPTION

Ultrasound devices may be used to perform diagnostic imaging and/or treatment, using sound waves with frequencies that are higher with respect to those audible to humans. Ultrasound imaging may be used to see internal soft tissue body structures, for example, to find a source of disease or to exclude any pathology. When pulses of ultrasound are transmitted into tissue (e.g., by using a probe), sound waves are reflected by the tissue, with different tissues reflecting varying degrees of sound. These reflected sound waves may then be recorded and displayed as an ultrasound image to the operator. The strength (e.g., amplitude) of the sound waves and the time it takes for the wave to travel through the body provide information used to produce the ultrasound image. Many different types of images can be formed using ultrasound devices, and the image formation may be performed in real time. For example, images can be generated that show two-dimensional cross-sections of tissue, blood flow, motion of tissue over time, the location of blood, the presence of specific molecules, the stiffness of tissue, and/or the anatomy of a three-dimensional region.

As described above, while sleep apnea may be treated using positive airway pressure devices, the delivery of pressure may be uncomfortable for the subject, and delivery of pressure may not always be necessary. Lack of subject comfort while using positive airway pressure devices may contribute to lack of subject compliance in treating sleep apnea with such devices.

In certain embodiments described herein, sleep apnea may be detected while a subject is sleeping, and that detection may trigger delivery of pressure to the subject. Detection of apnea may be accomplished using ultrasound imaging. For example, lung sliding is a sonographic identification of visceral pleura sliding on the parietal pleura lubrication by a small amount of pleural fluid as a subject breathes. The absence of lung sliding in ultrasound images of the lungs may be indicative of apnea. As another example, absence of movement of internal abdominal organs may be indicative of apnea.

The inventors have recognized that a wearable ultrasound patch device adhered to a subject may be used to collect ultrasound data for detecting apnea, and if apnea is detected, another device may be controlled to increase delivery of positive airway pressure to the subject. For example, the wearable ultrasound device may be worn on a subject's chest while sleeping in order to collect ultrasound data for detecting the absence of lung sliding, which may indicate sleep apnea. As another example, the wearable ultrasound device may be worn on a subject's abdomen while sleeping in order to collect ultrasound data for detecting the absence of movement of internal abdominal organs, which may indicate sleep apnea. The wearable ultrasound device may transmit ultrasound data to a processing device configured to determine if the ultrasound data indicates apnea. The processing device may use a statistical model (e.g., a deep learning statistical model) to determine whether the ultrasound data indicates apnea. If the processing device determines that the ultrasound data indicates apnea, the processing device may transmit an activation signal to a positive airway pressure device to increase pressure delivered to the subject, which may help to alleviate the apnea. When apnea is not detected, the pressure device may decrease the pressure delivered to the subject. This may help to maintain subject comfort while using the pressure device and increase subject compliance in treating sleep apnea with such devices.

It should be appreciated that the embodiments described herein may be implemented in any of numerous ways. Examples of specific implementations are provided below for illustrative purposes only. It should be appreciated that these embodiments and the embodiments provided above may be used individually, all together, or in any combination of two or more, as aspects of the technology described herein are not limited in this respect.

As referred to herein, determining that ultrasound data indicates apnea should be understood to mean that the determination is performed directly on the ultrasound data itself or on data generated from the ultrasound data. In other words, determining that second ultrasound data, which is generated from first ultrasound data, indicates apnea may be considered to mean that the first ultrasound data indicates apnea as well.

As referred to herein, delivering pressure to a subject may include delivering air having a certain pressure above atmospheric pressure to one of the subject's airways.

As referred to herein, any “processing circuitry” may include one or more processors.

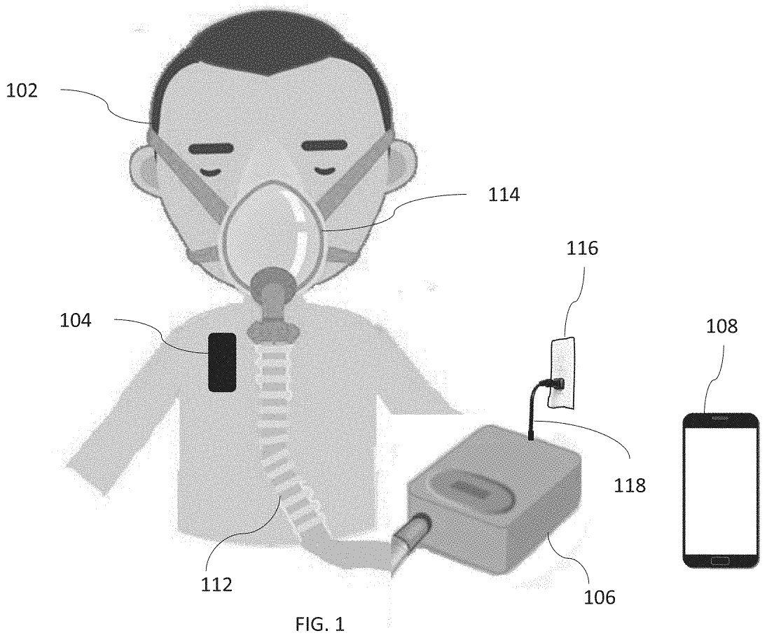

FIG. 1 illustrates an example system for delivering pressure to a subject's airway based on ultrasound data, in accordance with certain embodiments described herein. FIG. 1 illustrates a subject 102, an ultrasound device 104, a positive airway pressure device 106, tubing 112, a mask 114, a processing device 108, a power connector 118, and a power source 116. The mask 114 is coupled over the mouth and nose of the subject 102 (i.e., entrances to airways of the subject 102). The mask 114 is coupled to the tubing 112, and the tubing 112 is coupled to the positive airway pressure device 106. The power connector 118 is coupled both to the positive airway pressure device 106 and the power source 116 in order to supply the positive airway pressure device 106 with power. The ultrasound device 104 may be in communication with the processing device 108 (e.g., over a wireless connection, such as a BLUETOOTH, ZIGBEE, and/or WiFi wireless network, or over a wired connection, such as over a Universal Serial Bus (USB) cable or a Lightning cable). The processing device 108 may be in communication with the positive airway pressure device 106 (e.g., over a wireless network or over a wired connection).

The ultrasound device 104 may be wearable. In the illustrative embodiment of FIG. 1, the ultrasound device 104 is a patch configured to couple (e.g., with adhesive) to the skin of the subject 102. In FIG. 1, the ultrasound device 104 is coupled to the chest area of the subject 102. The ultrasound device 104 may collect ultrasound data from the subject 102. For example, the ultrasound data may be raw acoustical data, scan lines generated from collected raw acoustical data, and/or one or more ultrasound images generated from collected raw acoustical data. The ultrasound data may include ultrasound data collected from one or two of the subject 102's lungs. The ultrasound device 104 may transmit the ultrasound data (e.g., raw acoustical data, scan lines, and/or one or more ultrasound images) to the processing device 108.

The processing device 108 may be configured to determine whether the ultrasound data indicates apnea. For example, the processing device may be configured to determine whether the ultrasound data indicates an absence of lung sliding and/or an absence of movement of internal abdominal organs. As referred to herein, absence of lung sliding and absence of movement of internal abdominal organs may include lung sliding and movement of internal abdominal organs that is below a threshold amount. In some embodiments, the processing device 108 may perform this determination directly on the ultrasound data received from the ultrasound device 104. In some embodiments, the processing device 108 may perform this determination on ultrasound data generated from the ultrasound data received from the ultrasound device 104. For example, the processing device 108 may perform this determination on one or more ultrasound images generated from raw acoustical data and/or scan lines received from the ultrasound device 104, or the processing device 108 may perform this determination on scan lines generated from raw acoustical data received from the ultrasound device 104. In some embodiments, determining whether the ultrasound data indicates apnea or not may include determining whether the subject is experiencing apnea or not.

In some embodiments, to determine whether the ultrasound data indicates apnea, the processing device 108 may be configured to input the ultrasound data to a statistical model (e.g., a convolutional neural network or other deep learning model) trained to determine whether inputted ultrasound data indicates apnea. For example, the statistical model may determine whether inputted ultrasound indicates apnea by determining whether the ultrasound data indicates an absence of lung sliding or an absence of movement of internal abdominal organs. The statistical model may be trained with training data that includes ultrasound data labeled (e.g., manually by an annotator) with whether the ultrasound data indicates apnea. The statistical model may be stored on memory on the processing device 108, or the statistical model may be stored on memory on a remote server, and the processing device 108 may be configured to transmit the ultrasound data to the remote server and receive the output of the statistical model from the remote server. The statistical model may be a convolutional neural network, a fully connected neural network, a recurrent neural network (e.g., a long short-term memory (LSTM) recurrent neural network), a random forest, a support vector machine, a linear classifier, and/or any other statistical model. If the processing device 108 determines that the ultrasound data indicates apnea, the processing device 108 may transmit (e.g., over a wireless connection or over a wired connection) an activation signal to the positive airway pressure device 106.

In some embodiments, the positive airway pressure device 106 may include communication circuitry for receiving the activation signal from the processing device 108. For example, the positive airway pressure device 106 may include a data connector port (not shown in FIG. 1) such as a female USB port for receiving a USB cable connected to the processing device 108 and over which the positive airway pressure device 106 may receive the activation signal. As another example, the positive airway pressure device 106 may include wireless communication circuitry for communication over a wireless network, and the positive airway pressure device 106 may receive the activation signal over the wireless network.

In some embodiments, upon receiving the activation signal from the processing device 108, the positive airway pressure device 106 may increase pressure being delivered to the subject 102's airway. For example, the positive airway pressure device 106 may be off (i.e., pressure being delivered to the subject may be zero), and the activation signal may cause the positive airway pressure device 106 to turn on. The positive airway pressure device 106 turning on may cause a fan within the positive airway pressure device 106 to deliver pressure to the subject 102's airway (e.g., to increase the pressure being delivered to the subject 102's airway from zero). As another example, the positive airway pressure device 106 may be on, but a fan within the positive airway pressure device 106 may be off, and the activation signal may cause the fan to turn on and deliver pressure to the subject 102's airway. The pressure may be increased to a default value, or feedback systems (e.g., proportional-integral-derivative controllers) may be used to continuously modulate the pressure based on real-time ultrasound data. The delivery of pressure may be performed while the subject 102 is sleeping, and may help to alleviate the apnea indicated by the ultrasound data. For example, the pressure may help to open a closure in the subject's airways. The positive airway pressure device 106 may deliver the pressure from the positive airway pressure device 106 (e.g., using a fan in the positive airway pressure device 106), through the tubing 112, into the mask 114, and from the mask 114 into the subject's airway (e.g., through the mouth or nose).

In some embodiments, the activation signal may trigger the positive airway pressure device 106 to indefinitely deliver pressure to the subject 102 at the increased pressure. In such embodiments, the processing device 108 may be further configured to determine that ultrasound data received from the ultrasound device 104 does not indicate apnea. For example, the processing device 108 may be configured to determine that the ultrasound data indicates lung sliding. As another example, the processing device 108 may be configured to determine that the ultrasound data indicates movement of internal abdominal organs. In some embodiments, the processing device 108 may be configured to determine that the ultrasound data indicates both lung sliding and movement of internal abdominal organs. In such embodiments, the processing device 108 may transmit (e.g., over a wireless connection or a wired connection) a deactivation signal to the positive airway pressure device 106. Upon receiving the deactivation signal, the positive airway pressure device 106 may be configured to decrease the pressure being delivered to the subject 102 (e.g., to zero pressure). As the subject 102 may no longer be experiencing apnea, delivery of pressure (or delivery of a certain pressure) may no longer be necessary, and it may be helpful to cease to deliver pressure to the subject 102, or decrease the pressure, as delivery of pressure may be uncomfortable for the subject 102. In some embodiments, the activation signal may trigger the positive airway pressure device 106 to increase the pressure being delivered to the subject's airway for a set period of time. After the set period of time, the positive airway pressure device 106 may automatically decrease the pressure being delivered to the subject's airway (e.g., to zero pressure).

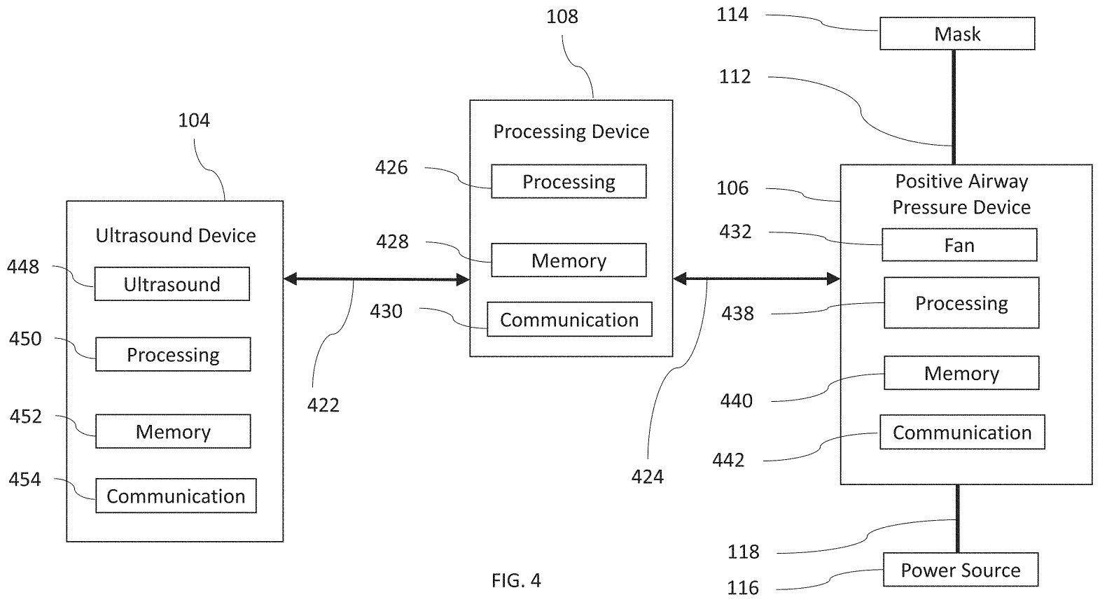

In some embodiments, rather than the processing device 108 determining whether the ultrasound data indicates apnea or does not indicate apnea, the ultrasound device 104 may determine whether the ultrasound data indicates apnea or does not indicate apnea. In such embodiments, the ultrasound device 104 may be in communication with the positive airway pressure device 106 (e.g., over a wireless connection or over a wired connection) and may transmit the activation signal to the positive airway pressure device 106. In some embodiments, rather than the processing device 108 determining whether the ultrasound data indicates apnea or does not indicate apnea, the positive airway pressure device 106 may determine whether the ultrasound data indicates apnea or does not indicate apnea. In such embodiments, the ultrasound device 104 may be in communication with the positive airway pressure device and may transmit the ultrasound data to the positive airway pressure device 106. Furthermore, in such embodiments, the positive airway pressure device 106 may trigger itself to increase or decrease the pressure delivered to the subject's airway (i.e., without generation of an activation signal by an external device). In embodiments in which the ultrasound device 104 or the positive airway pressure device 106 determines whether the ultrasound data indicates apnea or does not indicate apnea, the processing device 108 may be absent. In embodiments in which activation and deactivation signals are transmitted over a wired connection, the signal may be a communication according to a standard protocol (e.g., in the case of USB communication), or the signal may be a voltage transmitted over a wire that triggers the positive airway pressure device 106 (e.g., triggers a switch, relay, potentiometer, etc., that controls operation of the fan in the positive airway pressure device 106). Further description of the system illustrated in FIG. 1 may be found below with reference to FIG. 4.

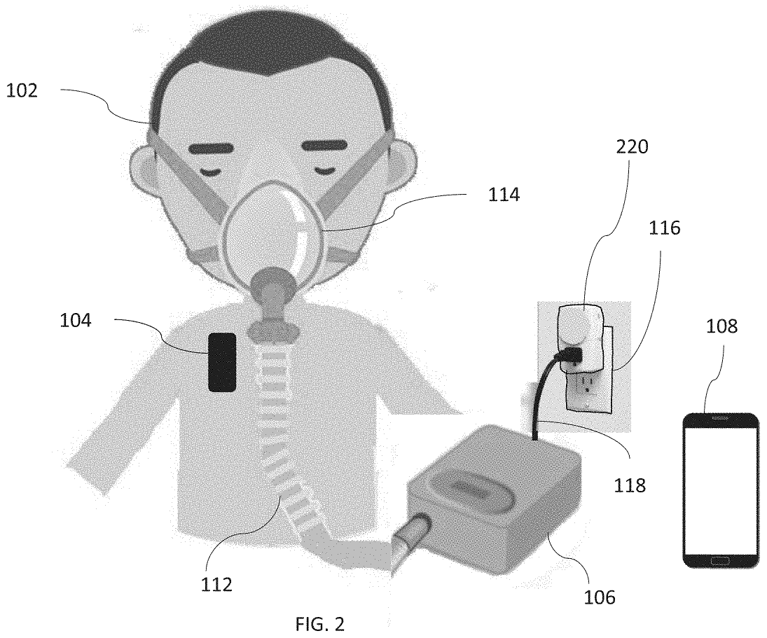

FIG. 2 illustrates another example system for delivering pressure to a subject based on ultrasound data, in accordance with certain embodiments described herein. The system of FIG. 2 differs from the system of FIG. 1 in that the system of FIG. 2 includes an adapter 220, and the adapter 220 is coupled both to the power source 116 and the power connector 118. In this embodiment, the positive airway pressure device 106 is not directly coupled to the power source 116, may not include communication circuitry, and may not receive an activation or deactivation signal (in contrast to the embodiment on FIG. 1). Instead, the adapter 220 may include communication circuitry and receive an activation or deactivation signal. For example, the adapter 220 may include a data connector port (not shown in FIG. 2) such as a female USB port for receiving a USB cable connected to the processing device 108 and over which the adapter 220 may receive the activation signal. As another example, the adapter 220 may include wireless communication circuitry for communication over a wireless network, and the adapter 220 may receive the activation signal over the wireless network.

Upon receiving the activation signal, the adapter 220 may route power from the power source 116 to the positive airway pressure device 106 through the power connector 118. This may turn on the positive airway pressure device 106 and initiate delivery of pressure from the positive airway pressure device 106 to the subject's airway. For example, the positive airway pressure device 106, upon connection to the power source 116, may turn on in a state in which a fan within the positive airway pressure device 106 is on and delivers pressure (i.e., increases pressure delivered to the subject's airway from zero).

In some embodiments, the activation signal may trigger the adapter 220 to indefinitely route power from the power source 116 to the positive airway pressure device 106. This may in turn cause the positive airway pressure device 106 to indefinitely deliver pressure to the subject at the increased pressure. In such embodiments, upon receiving the deactivation signal, the adapter 220 may be configured to cease to route power from the power source 116 to the positive airway pressure device 106. Cutting off power to the positive airway pressure device 106 may cause the positive airway pressure device 106 to turn off and cease to deliver pressure to the subject. As the subject 102 may no longer be experiencing apnea, delivery of pressure may no longer be necessary, and it may be helpful to cease to deliver pressure to the subject 102, as delivery of pressure may be uncomfortable for the subject 102. In some embodiments, the activation signal may trigger the adapter 220 to route power from the power source 116 to the positive airway pressure device 106 for a set period of time. After the set period of time, the adapter 220 may cease to route power from the power source 116 to the positive airway pressure device 106.

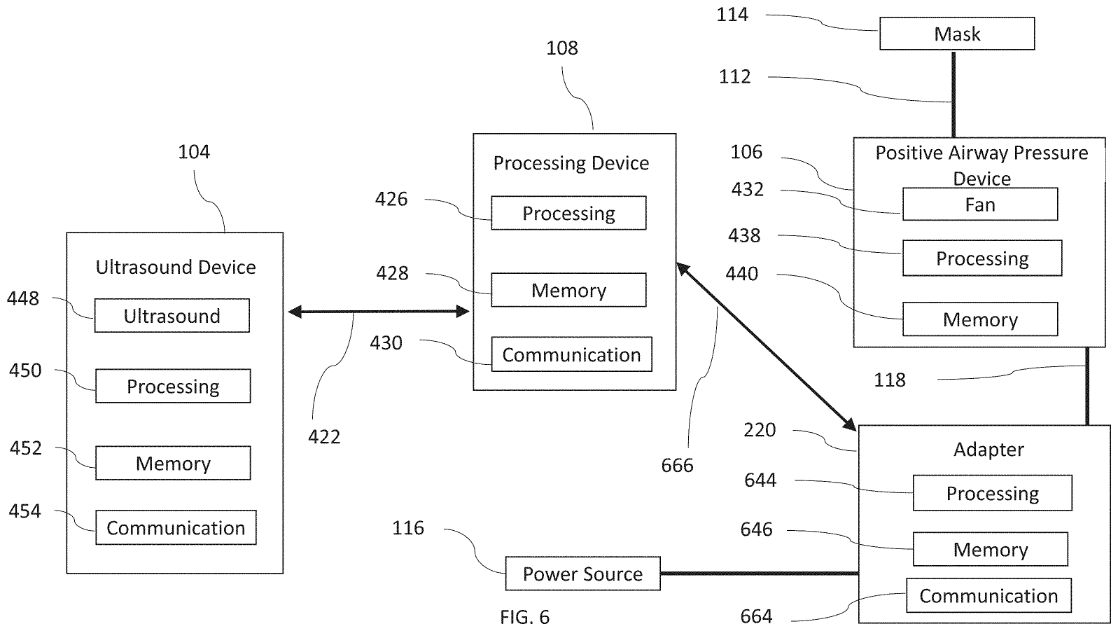

In some embodiments, rather than the processing device 108 determining whether the ultrasound data indicates apnea or does not indicate apnea, the ultrasound device 104 may determine whether the ultrasound data indicates apnea or does not indicate apnea. In such embodiments, the ultrasound device 104 may be in communication with the adapter 220 (e.g., over a wireless connection or over a wired connection), and may transmit the activation signal to the adapter 220. In some embodiments, rather than the processing device 108 determining whether the ultrasound data indicates apnea or does not indicate apnea, the adapter 220 may determine whether the ultrasound data indicates apnea or does not indicate apnea. In such embodiments, the ultrasound device 104 may transmit the ultrasound data to the adapter 220. Furthermore, in such embodiments, the adapter 220 may trigger itself to route or not route power from the power source 116 to the positive airway pressure device 106. In embodiments in which the ultrasound device 104 or the adapter 220 determines whether the ultrasound data indicates apnea or does not indicate apnea, the processing device 108 may be absent. In embodiments in which activation and deactivation signals are transmitted over a wired connection, the signal may be a communication according to a standard protocol (e.g., in the case of USB communication), or the signal may a voltage transmitted over a wire that triggers the adapter 220 (e.g., triggers a switch that controls routing of power from the power source 116 to the positive airway pressure device 106). Using the adapter 220 to control delivery of pressure to the subject 102 rather than the positive airway pressure device 106 itself may be helpful because it may enable standard positive airway pressure devices 106, which were not designed to be controlled by ultrasound data, to be controlled by ultrasound data due to augmentation with the adapter 220. Further description of the system illustrated in FIG. 2 may be found below with reference to FIG. 6.

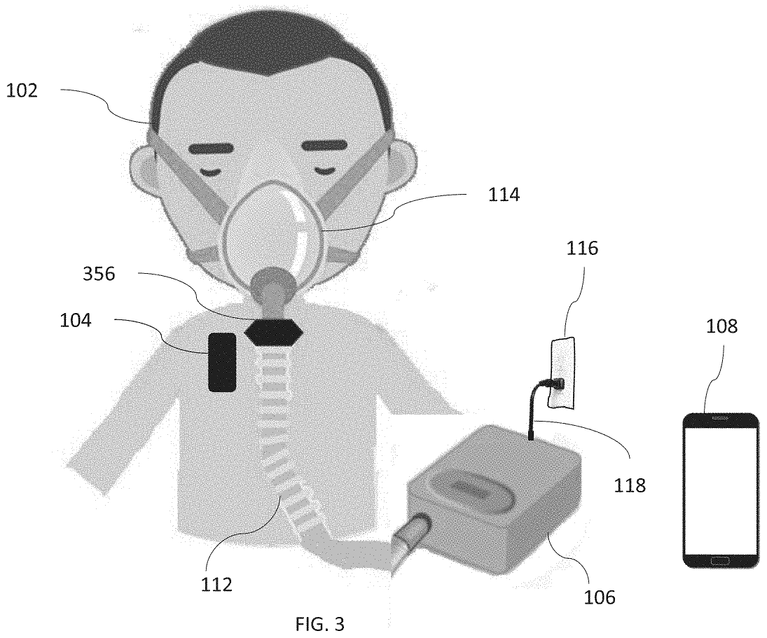

FIG. 3 illustrates another example system for delivering pressure to a subject based on ultrasound data, in accordance with certain embodiments described herein. The system of FIG. 3 differs from the system of FIG. 1 in that the system of FIG. 3 includes a valve 356, and the valve 356 is coupled to the tubing 112 between the positive airway pressure device 106 and the mask 114. The valve 356 may be an electronically-controlled valve configured to control air flow through the tubing 112. In this embodiment, the positive airway pressure device 106 may not include communication circuitry and may not receive an activation or deactivation signal (in contrast to the embodiment on FIG. 1). Instead, the valve 356 may include communication circuitry and receive an activation or deactivation signal. For example, the valve 356 may include a data connector port (not shown in FIG. 3) such as a female USB port for receiving a USB cable connected to the processing device 108 and over which the adapter 220 may receive the activation signal. As another example, the valve 356 may include wireless communication circuitry for communication over a wireless network, and the valve 356 may receive the activation signal over the wireless network.

Upon receiving the activation signal, the valve 356 may permit air to flow from the positive airway pressure device 106 to the mask 114 and subsequently to the subject's airway. As referred to herein, permitting air to flow should be understood to mean permitting more air to flow than was previously flowing (whether air was previously flowing or not). In other words, permitting air to flow from the positive airway pressure device 106 to the subject's airway may include the valve 356 opening from a fully closed state to a fully open state, from a fully closed state to a partially open state, from a partially closed state to a fully open state, or from a partially closed state to a partially open state. In any of these cases, the pressure being delivered from the positive airway pressure device 106 to the subject's airway may be increased (either from zero or from a non-zero pressure). The valve may be open to a default openness, or feedback systems (e.g., proportional-integral-derivative controllers) may be used to continuously modulate the openness of the valve based on real-time ultrasound data.

In some embodiments, the activation signal may trigger the valve 356 to indefinitely permit air to flow from the positive airway pressure device 106 to the subject's airway. This may in turn cause the positive airway pressure device 106 to indefinitely deliver pressure to the subject at the increased pressure. In such embodiments, upon receiving the deactivation signal, the valve 356 may be configured to prevent air from flowing from the positive airway pressure device 106 to the mask 114. As referred to herein, preventing air flow from flowing should be understood to mean preventing certain air from flowing that was previously flowing (whether all the air is prevented from flowing or only a portion of the air that was previously flowing is prevent from flowing). In other words, preventing air from flowing from the positive airway pressure device 106 to the subject's airway may include the valve 356 closing from a fully open state to a partially open state, from a fully open state to a fully closed state, from a partially open state to a fully closed state, or from a partially open state to a partially closed state. In any of these cases, the pressure being delivered from the positive airway pressure device 106 to the subject's airway may be decreased (either from zero or from a non-zero pressure). As the subject 102 may no longer be experiencing apnea, delivery of pressure (or delivery of pressure at a certain pressure) may no longer be necessary, and it may be helpful to cease to deliver pressure to the subject 102, or decrease the pressure, as delivery of pressure may be uncomfortable for the subject 102. In some embodiments, the activation signal may trigger the valve 356 to permit air to flow from the positive airway pressure device 106 to the subject's airway for a set period of time. After the set period of time, the valve 356 may prevent air from flowing from the positive airway pressure device 106 to the subject 102's airway.

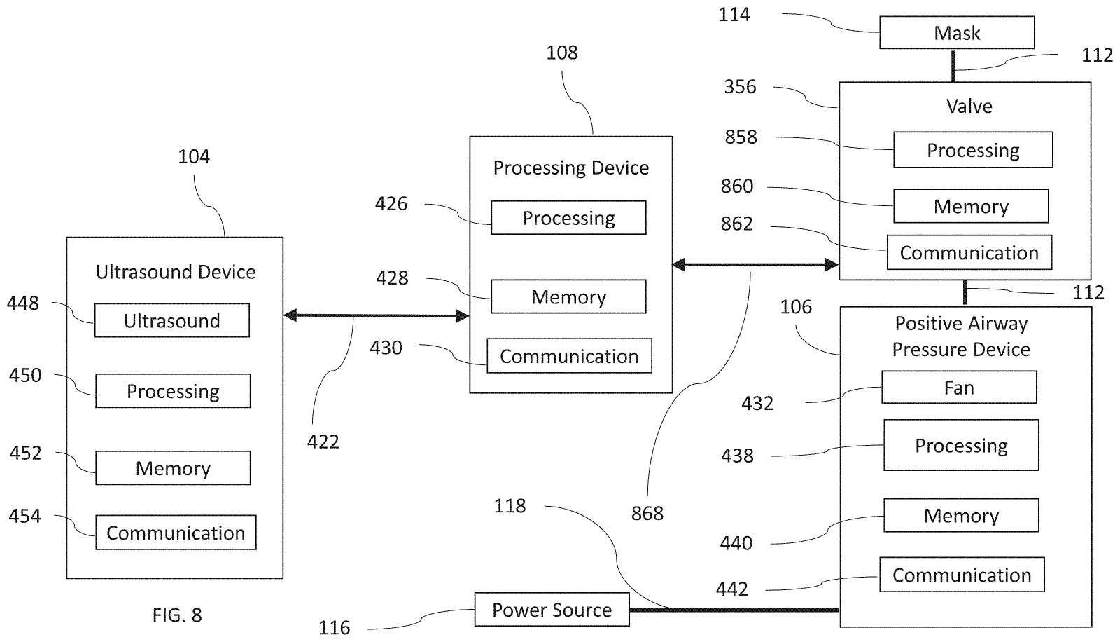

In some embodiments, rather than the processing device 108 determining whether the ultrasound data indicates apnea or does not indicate apnea, the ultrasound device 104 may determine whether the ultrasound data indicates apnea or does not indicate apnea. In such embodiments, the ultrasound device 104 may be in communication with the valve 356 (e.g., over a wireless connection or over a wired connection), and may transmit the activation signal to the valve 356. In some embodiments, rather than the processing device 108 determining whether the ultrasound data indicates apnea or does not indicate apnea, the valve 356 may determine whether the ultrasound data indicates apnea or does not indicate apnea. In such embodiments, the ultrasound device 104 may transmit the ultrasound data to the valve 356. Furthermore, in such embodiments, the valve 356 may trigger itself to permit or prevent air from flowing from the positive airway pressure device 106 to the subject 102's airway. In embodiments in which the ultrasound device 104 or the valve 356 determines whether the ultrasound data indicates apnea or does not indicate apnea, the processing device 108 may be absent. In embodiments in which activation and deactivation signals are transmitted over a wired connection, the signal may be a communication according to a standard protocol (e.g., in the case of USB communication), or the signal may be a voltage transmitted over a wire that triggers the valve 356 (e.g., triggers a switch, relay, potentiometer, etc., that controls closure and opening of the valve 356). Using the valve 356 to control delivery of pressure to the subject 102 rather than using the positive airway pressure device 106 itself may be helpful because it may enable standard positive airway pressure devices 106, which were not designed to be controlled by ultrasound data, to be controlled by ultrasound data due to augmentation with the valve 356. Furthermore, control by the valve 356 may enable a fan within the positive airway pressure device 106 to remain on even when pressure is not being delivered to the subject 102, which may reduce noise disturbance due to the fan turning on when delivery of pressure is needed. Further description of the system illustrated in FIG. 3 may be found below with reference to FIG. 8.

In the illustrative embodiments of FIGS. 1-3, the ultrasound device 104 is coupled to the chest area of the subject 102 in order to collect ultrasound data regarding lung sliding. The absence of lung sliding in ultrasound data may be indicative of apnea. However, the ultrasound device 104 may also be coupled to other areas of the subject 102 from which ultrasound data indicative of apnea may be collected. For example, the ultrasound device 104 may be coupled to the abdomen in order to collect ultrasound data regarding movement of internal abdominal organs.

FIG. 4 is a schematic block diagram of an example system for delivering pressure to a subject based on ultrasound data, in accordance with certain embodiments described herein. The system shown in FIG. 4 may correspond to the system shown in FIG. 1. As shown, the system includes an ultrasound device 104, a processing device 108, a positive airway pressure device 106, a mask 114, tubing 112, a power source 116, a communication link 422, and a communication link 424. The ultrasound device 104 includes ultrasound circuitry 448, processing circuitry 450, memory circuitry 452, and communication circuitry 454. The processing device 108 includes processing circuitry 426, memory circuitry 428, and communication circuitry 430. The positive airway pressure device 106 includes a fan 432, processing circuitry 438, memory circuitry 440, and communication circuitry 442. The ultrasound device 104 is configured to communicate with the processing device 108 over the communication link 422. The communication link 422 may include a wired connection and/or a wireless connection. The processing device 108 is configured to communicate with the positive airway pressure device 106 over the communication link 424. The communication link 424 may include a wired connection and/or a wireless connection. The positive airway pressure device 106 is connected to the power source 116 by the power connector 118. The mask 114 is connected to the positive airway pressure device 106 by the tubing 112.

The ultrasound device 104 may be configured to generate ultrasound data. The ultrasound data may be employed to generate an ultrasound image, for example. The ultrasound device 104 may be constructed in any of a variety of ways. In some embodiments, the ultrasound device 104 may include a waveform generator that transmits a signal to a transmit beamformer which in turn drives transducer elements within a transducer array to emit pulsed ultrasonic signals into a structure, such as a subject. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes may be sent to a receive beamformer that outputs ultrasound data.

The ultrasound circuitry 448 may be configured to generate the ultrasound data. The ultrasound circuitry 448 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 448 (e.g., transmit circuitry, receive circuitry, control circuitry, power management circuitry, and processing circuitry) to form a monolithic ultrasound device.

The processing circuitry 450 may control operation of the ultrasound device 104, and in particular, operation of the ultrasound circuitry 448, the memory circuitry 452, and the communication circuitry 454. As one example, the processing circuitry 450 may control collection of ultrasound data by the ultrasound device 104. The memory circuitry 452 may include non-transitory computer-readable storage media. The processing circuitry 450 may control writing data to and reading data from the memory circuitry 452 in any suitable manner. To perform any of the functionality of the ultrasound device 104 described herein, the processing circuitry 450 may execute one or more processor-executable instructions stored in one or more non-transitory computer-readable storage media (e.g., the memory circuitry 452), which may serve as non-transitory computer-readable storage media storing processor-executable instructions for execution by the processing circuitry 450. The communication circuitry 454 may be configured to enable communication between the ultrasound device 104 and the processing device 108 over the communication link 422. The communication circuitry 454 may include an antenna and circuitry capable of transmitting and receiving signals according to a certain wireless communication protocol (e.g., WiFi, BLUETOOTH, or Zigbee) and/or a data connector port for accepting a data connector of a particular type and circuitry capable of transmitting and receiving signals according to a certain protocol.

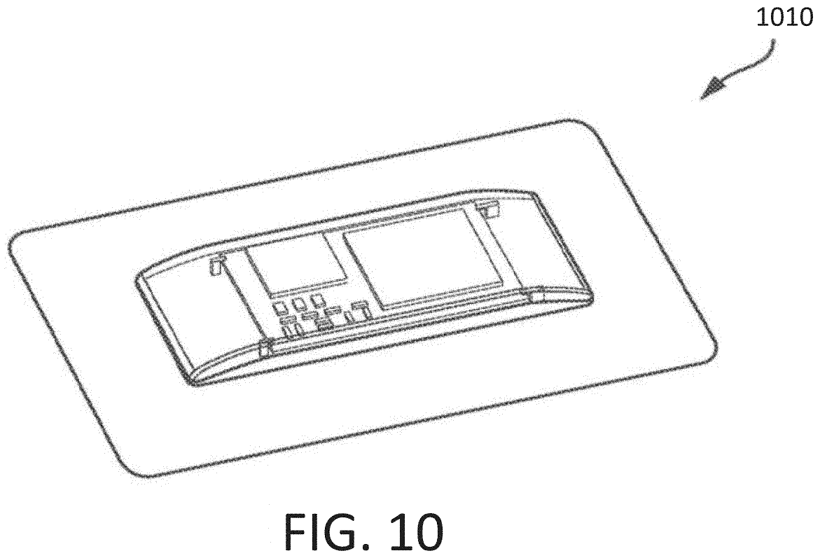

The ultrasound device 104 may be configured as a wearable ultrasound device, such as a patch. For further discussion of ultrasound devices and systems, such as more detail of components that may be included in the ultrasound device 104, see U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application). Wearable ultrasound devices are described further below with reference to FIGS. 10-15

The processing device 108 may be configured to process ultrasound data from the ultrasound device 104 to generate ultrasound images. The processing may be performed by, for example, the processing circuitry 426. The processing circuitry 426 may also be adapted to control the acquisition of ultrasound data with the ultrasound device 104. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing circuitry 426 of the processing device 108 may also be configured to control operation of the processing device 108. The processing circuitry 426 may be configured to control operation of the memory circuitry 428 and the communication circuitry 430. The memory circuitry 428 may include non-transitory computer-readable storage media. The processing circuitry 426 may control writing data to and reading data from the memory circuitry 428 in any suitable manner. To perform any of the functionality of the processing device 108 described herein, the processing circuitry 426 may execute one or more processor-executable instructions stored in one or more non-transitory computer-readable storage media (e.g., the memory circuitry 428), which may serve as non-transitory computer-readable storage media storing processor-executable instructions for execution by the processing circuitry 426.

The communication circuitry 430 may be configured to enable communication between the processing device 108 and the ultrasound device 104 over the communication link 42, and between the processing device 108 and the positive airway pressure device 106 over the communication link 424. When the communication circuitry 430 is configured for wired communication, the communication circuitry 430 may include a data connector port for accepting a data connector of a particular type and circuitry capable of transmitting and receiving signals according to a certain protocol. For example, in the case of USB communication, the communication circuitry 430 may include a female USB port and circuitry capable of communication according to the USB protocol. When the communication circuitry 430 is configured for wireless communication, the communication circuitry 430 may include an antenna and circuitry capable of transmitting and receiving signals according to a certain protocol. In some embodiments, the communication circuitry 430 may include circuitry for communication according to multiple protocols and/or circuitry for wired and wireless communication. In some embodiments, the communication link 422 and the communication link 424 may be different types of communication links. In other words, the processing device 108 may communicate with the ultrasound device 104 and the positive airway pressure device 106 using different types of communication links. For example, the processing device 108 may communicate with the ultrasound device 104 using WiFi and with the positive airway pressure device 106 using BLUETOOTH. In some embodiments, the communication link 422 may be a wireless communication link and the communication link 424 may be a wired communication link. For example, the processing device 108 may communicate with the ultrasound device 104 using WiFi and with the positive airway pressure device 106 using a USB connection.

The processing circuitry 426 may be configured to receive ultrasound data from the ultrasound device 104 over the communication link 422 using the communication circuitry 430, and to determine that ultrasound data indicates apnea. Based on this determination, the processing circuitry 426 may be configured to transmit an activation signal to the positive airway pressure device 106 over the communication link 424 using the communication circuitry 430. The processing circuitry 426 may also be configured to determine that ultrasound data does not indicate apnea. Based on this determination, the processing circuitry 426 may be configured to transmit a deactivation signal to the positive airway pressure device 106 over the communication link 424 using the communication circuitry 430.

It should be appreciated that the processing device 108 may be implemented in any of a variety of ways. For example, the processing device 108 may be implemented as a handheld device such as a mobile smartphone or a tablet. Thereby, an operator of the ultrasound device 104 may be able to operate the ultrasound device 104 with one hand and hold the processing device 108 with another hand. In other examples, the processing device 108 may be implemented as a portable device that is not a handheld device such as a laptop. In yet other examples, the processing device 108 may be implemented as a stationary device such as a desktop computer.

The positive airway pressure device 106 may be any kind of positive airway pressure device, such as a continuous positive airway pressure (CPAP) device, an automatic positive airway pressure (APAP) device, or a bilevel positive airway pressure (BiPAP) device. The fan 432 of the positive airway pressure device 106 may be configured to generate pressure. The tubing 112 may be configured to be substantially airtight and to convey the pressure from the fan 432 to the mask 114. The mask 114 may be configured to couple over the nose and mouth of a subject (e.g., using straps) in order to create a substantially airtight seal around the nose and mouth and to supply the pressure from the tubing 112 to the nose and/or mouth of the subject.

The processing circuitry 438 may control operation of the positive airway pressure device 106, which may include controlling operation of the fan 432, the memory circuitry 440, and the communication circuitry 442. For example, the processing circuitry 438 may control how much pressure is produced by the fan 432, and may control when the fan 432 produces the pressure. The memory circuitry 440 may include non-transitory computer-readable storage media. The processing circuitry 438 may control writing data to and reading data from the memory circuitry 440 in any suitable manner. To perform any of the functionality of the positive airway pressure device 106 described herein, the processing circuitry 438 may execute one or more processor-executable instructions stored in one or more non-transitory computer-readable storage media (e.g., the memory circuitry 440), which may serve as non-transitory computer-readable storage media storing processor-executable instructions for execution by the processing circuitry 438.

The communication circuitry 442 may be configured to enable communication between the positive airway pressure device 106 and the processing device 108 over the communication link 424. When the communication circuitry 442 is configured for wired communication, the communication circuitry 442 may include a data connector port for accepting a data connector of a particular type and circuitry capable of transmitting and receiving signals according to a certain protocol. For example, in the case of USB communication, the communication circuitry 442 may include a female USB port and circuitry capable of communication according to the USB protocol. When the communication circuitry 442 is configured for wireless communication, the communication circuitry 442 may include an antenna and circuitry capable of transmitting and receiving signals according to a certain protocol. The processing circuitry 438 of the positive airway pressure device 106 may be configured to receive, using the communication circuitry 442, activation and/or deactivation signals from the processing device 108, and to increase and/or decrease pressure generated by the fan 432 based on the activation and/or deactivation signals.

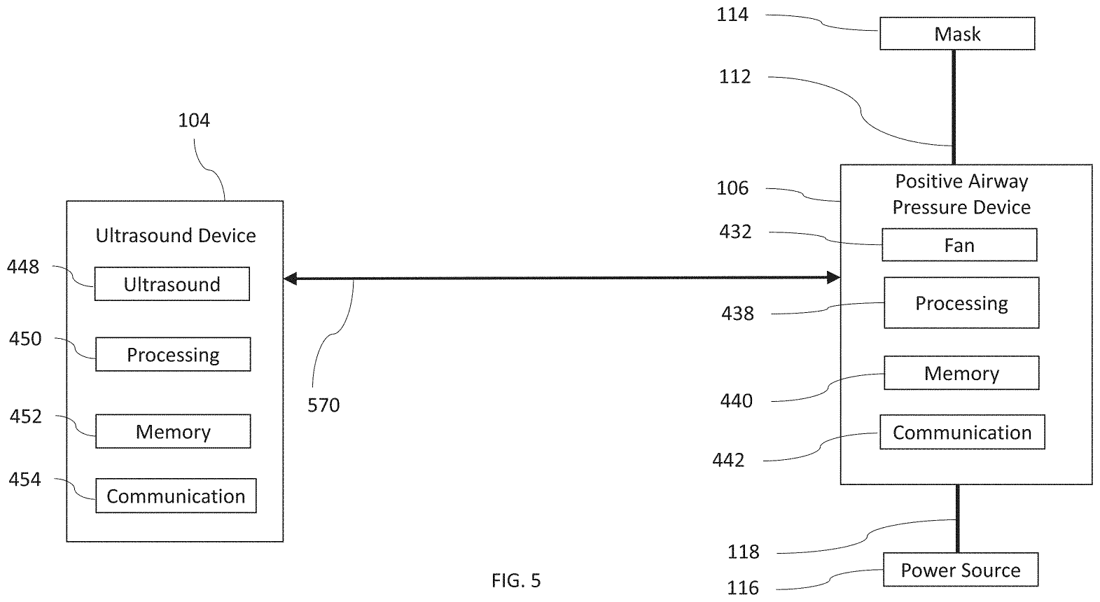

FIG. 5 is a schematic block diagram of another example system for delivering pressure to a subject based on ultrasound data, in accordance with certain embodiments described herein. The system of FIG. 5 differs from the system of FIG. 4 in that in the system of FIG. 5, the processing device 108 is absent, and the ultrasound device 104 communicates directly with the positive airway pressure device 106 over a communication link 570. In such embodiments, the processing circuitry 450 of the ultrasound device 104 may be configured to determine that ultrasound data indicates apnea. Based on this determination, the processing circuitry 450 may be configured to transmit an activation signal to the positive airway pressure device 106 over the communication link 570 using the communication circuitry 454 of the ultrasound device 104. The processing circuitry 450 may also be configured to determine that ultrasound data does not indicate apnea. Based on this determination, the processing circuitry 450 may be configured to transmit a deactivation signal to the positive airway pressure device 106 over the communication link 570 using the communication circuitry 454. Alternatively, the processing circuitry 438 of the positive airway pressure device 106 may be configured to receive ultrasound data from the ultrasound device 104 over the communication link 570 using the communication circuitry 442, and to determine that ultrasound data indicates apnea. Based on this determination, the processing circuitry 438 may be configured to increase pressure generated by the fan 432. The processing circuitry 438 may also be configured to determine that ultrasound data does not indicate apnea. Based on this determination, the processing circuitry 438 may be configured to decrease pressure generated by the fan 432. Further description of the operation of the system illustrated in FIG. 4 may be found with reference to FIG. 1.

FIG. 6 is a schematic block diagram of another example system for delivering pressure to a subject based on ultrasound data, in accordance with certain embodiments described herein. The system shown in FIG. 6 may correspond to the system shown in FIG. 2. The system of FIG. 6 differs from the system of FIG. 4 in that the system of FIG. 6 includes an adapter 220 and the positive airway pressure device 106 lacks the communication circuitry 442. The adapter 220 includes processing circuitry 644, memory circuitry 646, and communication circuitry 664. The processing device 108 and the adapter 220 may communicate over a communication link 666. The communication link 666 may include a wired connection and/or a wireless connection. The adapter 220 is connected to the power source 116, and the adapter 220 is also connected to the positive airway pressure device 106 through the power connector 118.

The processing circuitry 644 of the adapter 220 may control operation of the adapter 220, which may include controlling operation of the memory circuitry 646 and the communication circuitry 442. The memory circuitry 646 may include non-transitory computer-readable storage media. The processing circuitry 644 may control writing data to and reading data from the memory circuitry 646 in any suitable manner. To perform any of the functionality of the adapter 220 described herein, the processing circuitry 644 may execute one or more processor-executable instructions stored in one or more non-transitory computer-readable storage media (e.g., the memory circuitry 646), which may serve as non-transitory computer-readable storage media storing processor-executable instructions for execution by the processing circuitry 644.

The communication circuitry 664 may be configured to enable communication between the adapter 220 and the processing device 108 over the communication link 666. When the communication circuitry 664 is configured for wired communication, the communication circuitry 664 may include a data connector port for accepting a data connector of a particular type and circuitry capable of transmitting and receiving signals according to a certain protocol. For example, in the case of USB communication, the communication circuitry 664 may include a female USB port and circuitry capable of communication according to the USB protocol. When the communication circuitry 664 is configured for wireless communication, the communication circuitry 664 may include an antenna and circuitry capable of transmitting and receiving signals according to a certain protocol.

The processing circuitry 426 of the processing device 108 may be configured to receive ultrasound data from the ultrasound device 104 over the communication link 422 using the communication circuitry 430, and to determine that ultrasound data indicates apnea. Based on this determination, the processing circuitry 426 may be configured to transmit an activation signal to the adapter 220 over the communication link 666 using the communication circuitry 430. The processing circuitry 426 may also be configured to determine that ultrasound data does not indicate apnea. Based on this determination, the processing circuitry 426 may be configured to transmit a deactivation signal to the adapter 220 over the communication link 666 using the communication circuitry 430.

The processing circuitry 644 of the adapter 220 may be configured to receive, using the communication circuitry 664, activation and/or deactivation signals from the processing device 108, and to route power or prevent power from being routed from the power source 116 to the positive airway pressure device 106 based on the activation and/or deactivation signals. To route power or prevent power from being routed, the adapter 220 may be configured to open or close a switch between two terminals, one terminal connected to the power source 116, and the other terminal connected to the power connected 118.

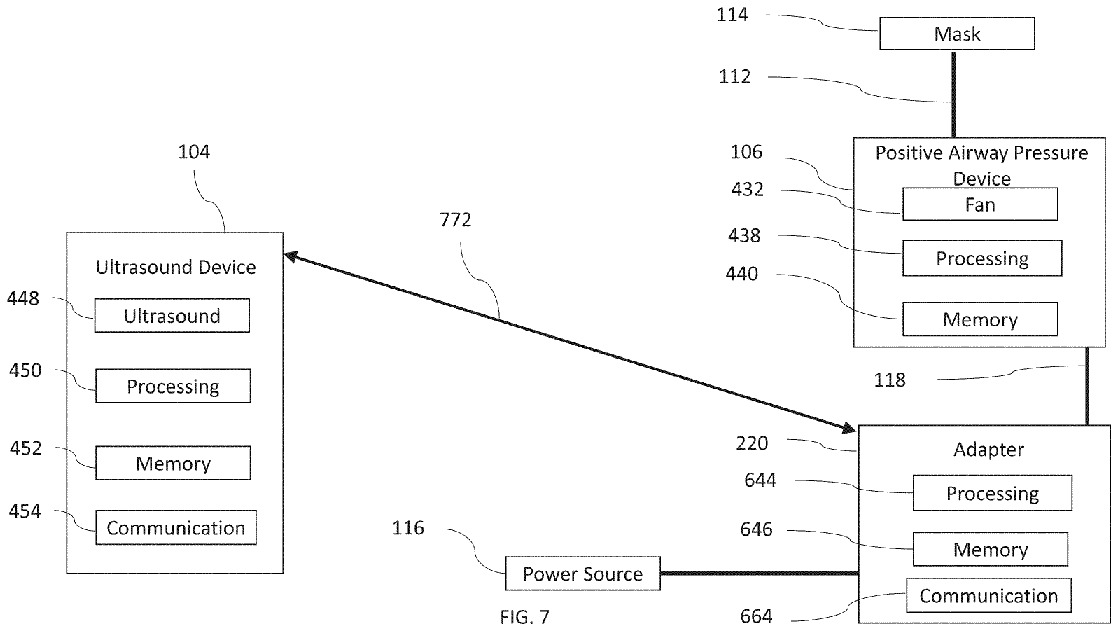

FIG. 7 is a schematic block diagram of another example system for delivering pressure to a subject based on ultrasound data, in accordance with certain embodiments described herein. The system of FIG. 7 differs from the system of FIG. 6 in that in the system of FIG. 7, the processing device 108 is absent, and the ultrasound device 104 communicates directly with the adapter 220 over a communication link 772. In such embodiments, the processing circuitry 450 of the ultrasound device 104 may be configured to determine that ultrasound data indicates apnea. Based on this determination, the processing circuitry 450 may be configured to transmit an activation signal to the adapter 220 over the communication link 772 using the communication circuitry 454 of the ultrasound device 104. The processing circuitry 450 may also be configured to determine that ultrasound data does not indicate apnea. Based on this determination, the processing circuitry 450 may be configured to transmit a deactivation signal to the adapter 220 over the communication link 772 using the communication circuitry 454. Alternatively, the processing circuitry 644 of the adapter 220 may be configured to receive ultrasound data from the ultrasound device 104 over the communication link 772 using the communication circuitry 664, and to determine that ultrasound data indicates apnea. Based on this determination, the processing circuitry 644 may be configured to route power from the power source 116 to the positive airway pressure device 106 through the power connector 118. The processing circuitry 644 may also be configured to determine that ultrasound data does not indicate apnea. Based on this determination, the processing circuitry 644 may be configured to prevent routing of power from the power source 116 to the positive airway pressure device 106 through the power connector 118. Further description of the operation of the system illustrates in FIG. 6 may be found with reference to FIG. 2.

FIG. 8 is a schematic block diagram of another example system for delivering pressure to a subject based on ultrasound data, in accordance with certain embodiments described herein. The system shown in FIG. 8 may correspond to the system shown in FIG. 3. The system of FIG. 8 differs from the system of FIG. 4 in that the system of FIG. 8 includes a valve 356 connected between the positive airway pressure device 106 and the mask 114. The valve 356 includes processing circuitry 858, memory circuitry 860, and communication circuitry 862. The processing device 108 and the valve 356 may communicate over a communication link 868. The communication link 868 may include a wired connection and/or a wireless connection.

The processing circuitry 858 of the valve 356 may control operation of the valve 356, which may include controlling operation of the memory circuitry 860 and the communication circuitry 862. The memory circuitry 646 may include non-transitory computer-readable storage media. The processing circuitry 644 may control writing data to and reading data from the memory circuitry 646 in any suitable manner. To perform any of the functionality of the adapter 220 described herein, the processing circuitry 644 may execute one or more processor-executable instructions stored in one or more non-transitory computer-readable storage media (e.g., the memory circuitry 646), which may serve as non-transitory computer-readable storage media storing processor-executable instructions for execution by the processing circuitry 644.

The communication circuitry 862 may be configured to enable communication between the valve 356 and the processing device 108 over the communication link 868. When the communication circuitry 862 is configured for wired communication, the communication circuitry 862 may include a data connector port for accepting a data connector of a particular type and circuitry capable of transmitting and receiving signals according to a certain protocol. For example, in the case of USB communication, the communication circuitry 862 may include a female USB port and circuitry capable of communication according to the USB protocol. When the communication circuitry 862 is configured for wireless communication, the communication circuitry 862 may include an antenna and circuitry capable of transmitting and receiving signals according to a certain protocol.

The processing circuitry 426 of the processing device 108 may be configured to receive ultrasound data from the ultrasound device 104 over the communication link 422 using the communication circuitry 430, and to determine that ultrasound data indicates apnea. Based on this determination, the processing circuitry 426 may be configured to transmit an activation signal to the adapter valve 356 over the communication link 666 using the communication circuitry 430. The processing circuitry 426 may also be configured to determine that ultrasound data does not indicate apnea. Based on this determination, the processing circuitry 426 may be configured to transmit a deactivation signal to the valve 356 over the communication link 666 using the communication circuitry 430.

The processing circuitry 858 of the valve 356 may be configured to receive, using the communication circuitry 862, activation and/or deactivation signals from the processing device 108, and to permit or prevent, based on the activation and/or deactivation signals, air from flowing from the positive airway pressure device 106 to the mask 114 through the tubing 112.

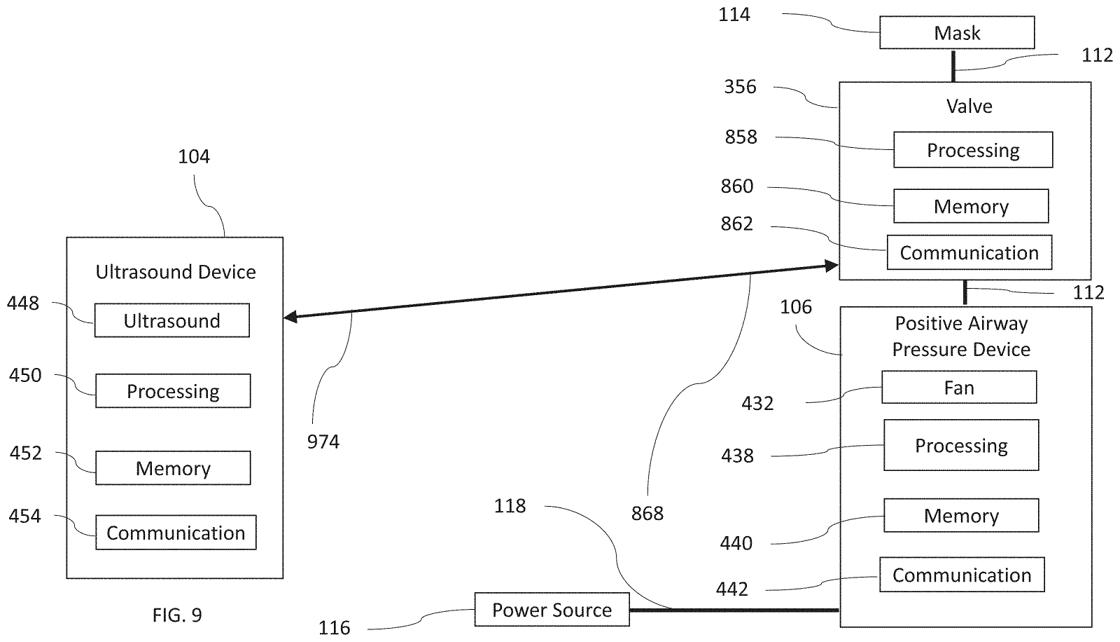

FIG. 9 is a schematic block diagram of another example system for delivering pressure to a subject based on ultrasound data, in accordance with certain embodiments described herein. The system of FIG. 9 differs from the system of FIG. 8 in that in the system of FIG. 9, the processing device 108 is absent, and the ultrasound device 104 communicates directly with the valve 356 over a communication link 974. In such embodiments, the processing circuitry 450 of the ultrasound device 104 may be configured to determine that ultrasound data indicates apnea. Based on this determination, the processing circuitry 450 may be configured to transmit an activation signal to the valve 356 over the communication link 974 using the communication circuitry 454 of the ultrasound device 104. The processing circuitry 450 may also be configured to determine that ultrasound data does not indicate apnea. Based on this determination, the processing circuitry 450 may be configured to transmit a deactivation signal to the valve 356 over the communication link 974 using the communication circuitry 454. Alternatively, the processing circuitry 858 of the valve 356 may be configured to receive ultrasound data from the ultrasound device 104 over the communication link 974 using the communication circuitry 862, and to determine that ultrasound data indicates apnea. Based on this determination, the processing circuitry 858 may be configured to permit air to flow from the positive airway pressure device 106 to the mask 114 through the tubing 112. The processing circuitry 858 may also be configured to determine that ultrasound data does not indicate apnea. Based on this determination, the processing circuitry 858 may be configured to prevent air from flowing from the positive airway pressure device 106 to the mask 114 through the tubing 112. Further description of the operation of the system illustrates in FIG. 8 may be found with reference to FIG. 3.

It should be appreciated that the positive airway pressure device 106 described above may be a positive airway pressure device not originally configured to operate based on ultrasound data, but may be augmented by the adapter 220 or the valve 356 to provide this functionality. It should also be appreciated that while systems described above include a positive airway pressure device having a fan, a mask, and tubing, other embodiments of positive airways devices may be used. For example, certain pressure devices may include a single body that is configured to be partially inserted into the subject's nose. Such a device may include micro-blowers configured to deliver pressure to the subject's nose, and may obviate the need for a mask that fits over the subject's nose and mouth and for separate tubing and a separate pressure device containing a fan. If these positive airway devices include communication circuitry (e.g., wireless communication circuitry) that allow them to be triggered by a processing device, they may also be used for triggering delivery of pressure in response to detecting that ultrasound data indicates apnea, as described above.

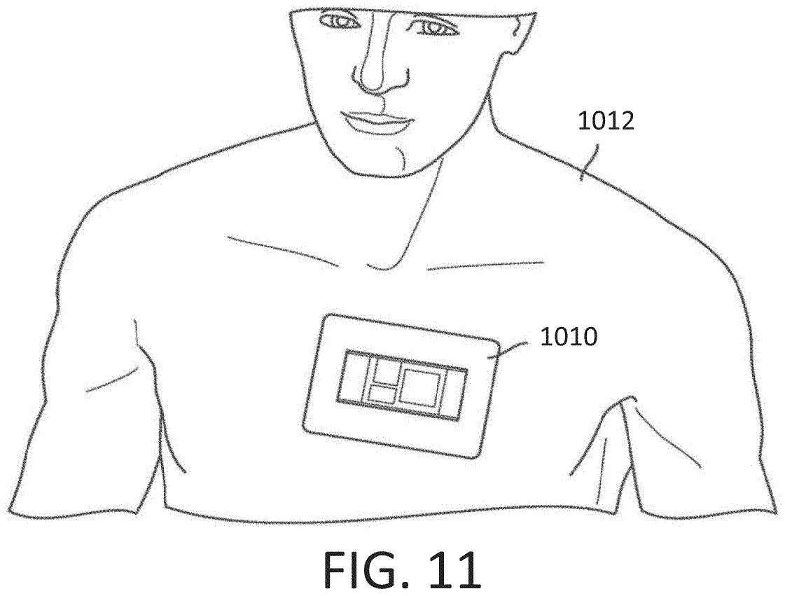

FIG. 10 illustrates an ultrasound patch 1010 and FIG. 11 illustrates the ultrasound patch 1010 coupled to a subject 1012 in accordance with certain embodiments described herein. The ultrasound patch 1010 may be configured to offload, for example wirelessly, data collected by the ultrasound patch 1010 to one or more external auxiliary devices (not shown) for further processing. For purposes of illustration, a top housing of the ultrasound patch 1010 is depicted in a transparent manner to depict exemplary locations of various internal components of the ultrasound patch.

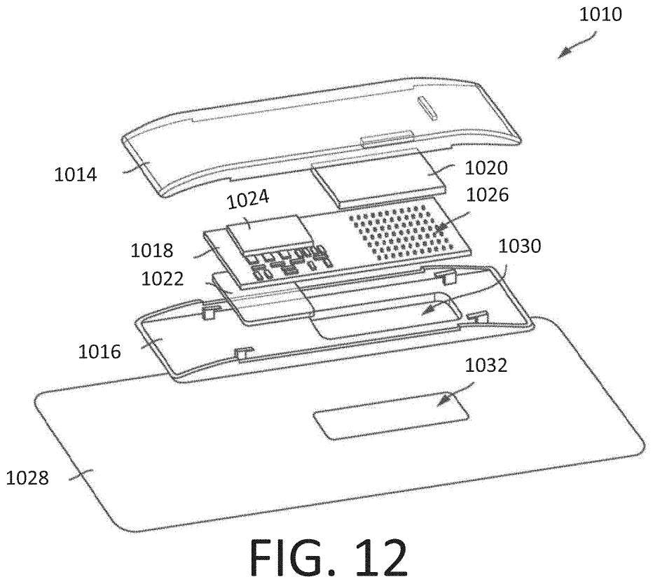

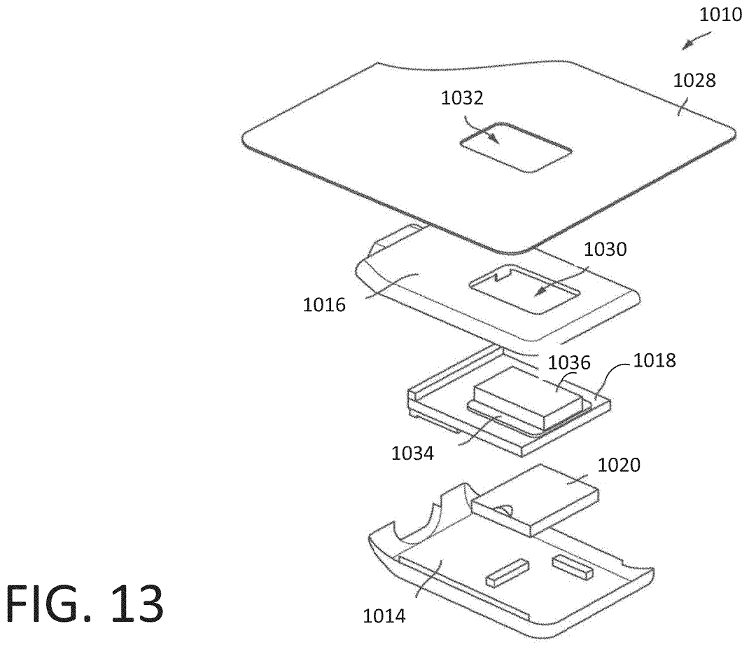

FIGS. 12 and 13 show exploded views of the ultrasound patch 1010 in accordance with certain embodiments described herein. As particularly illustrated in FIG. 12, the ultrasound patch 1010 includes an upper housing 1014, a lower housing 1016, and a circuit board 1018. The circuit board 1018 may be configured to support various components, such as for example a heat sink 1020, a battery 1022, and communications circuitry 1024. In one embodiment, the communication circuitry 1024 includes one or more short- or long-range communication platforms. Exemplary short-range communication platforms include Bluetooth (BT), Bluetooth Low Energy (BLE), and Near-Field Communication (NFC). Exemplary long-range communication platforms include WiFi and Cellular. While not shown, the communication circuitry 1024 may include front-end radio, antenna and other processing circuitry configured to communicate radio signal to an external auxiliary electronic device (not shown). The radio signal may include ultrasound imaging information obtained by the ultrasound patch 1010. In an exemplary embodiment, the communication platform transmits periodic beacon signals according to I10 802.11 and other prevailing standards. The beacon signal may include a BLE advertisement. Upon receipt of the beacon signal or the BLE advertisement, an external auxiliary device (not shown) may respond to the ultrasound patch 1010. That is, the response to the beacon signal may initiate a communication handshake between the ultrasound patch 1010 and the auxiliary device. The auxiliary device may include a laptop computer, a desktop computer, a smartphone, a tablet device, or any other device configured for wireless communication. The auxiliary device may act as a gateway to cloud or internet communication. In an exemplary embodiment, the auxiliary device may include the subject's own smart device (e.g., smartphone) which communicatively couples to the ultrasound patch 1010 and periodically receives ultrasound information from the ultrasound patch 1010. The auxiliary device may then communicate the received ultrasound information to external sources. In some embodiments, the ultrasound patch 1010 may offload ultrasound information to the auxiliary device in real-time.

The circuit board 1018 may include processing circuitry, including one or more controllers and/or field-programmable gate arrays (FPGAs) to direct communication through the communication circuitry 1024. For example, the circuit board 1018 may engage the communication circuitry 1024 periodically or on as-needed basis to communicate information with one or more auxiliary devices. Ultrasound information may include signals and information defining an ultrasound image captured by the ultrasound patch 1010. Ultrasound information may also include control parameters communicated from the auxiliary device to the ultrasound patch 1010. The control parameters may dictate the scope of the ultrasound data/image to be obtained by ultrasound patch 1010.

In one embodiment, the auxiliary device may store ultrasound information received from the ultrasound patch 1010. In another embodiment, the auxiliary device may relay ultrasound information received from the ultrasound patch 1010 to another station. For example, the auxiliary device may use WiFi to communicate the ultrasound information received from the ultrasound patch 1010 to a cloud-based server. The cloud-based server may be a hospital server or a server accessible to the physician directing ultrasound imaging. In another exemplary embodiment, the ultrasound patch 1010 may send sufficient ultrasound information to the auxiliary device such that the auxiliary device may construct an ultrasound image therefrom. In this manner, communication bandwidth and power consumption may be minimized at the ultrasound patch 1010.

In still another embodiment, the auxiliary device may engage the ultrasound patch 1010 through radio communication (i.e., through the communication circuitry 1024) to actively direct operation of the ultrasound patch 1010. For example, the auxiliary device may direct the ultrasound patch 1010 to produce ultrasound data from the subject at periodic intervals. The auxiliary device may direct the depth of the ultrasound images taken by the ultrasound patch 1010. In still another example, the auxiliary device may control the manner of operation of the ultrasound patch 1010 so as to preserve power consumption at the battery 1022. Upon receipt of ultrasound information from the ultrasound patch 1010, the auxiliary device may operate to cease imaging, increase imaging rate or communicate an alarm to the subject or to a third party (e.g., physician or emergency personnel).

As shown in FIG. 12, a plurality of through vias 1026 (e.g., copper) may be used for a thermal connection between the heat sink 1020 and one or more CMOS chips (not shown in FIG. 12). For example, the CMOS chip may be an application-specific integrated circuit (ASIC). The ASIC may be part of an ultrasound-on-a-chip (i.e., a device including micromachined ultrasound transducers integrated with an ASIC or other semiconductor die containing integrated circuitry). As further depicted in FIG. 12, the ultrasound patch 1010 may also include a dressing 1028 that provides an adhesive surface for both the ultrasound patch housing as well as to the skin of a subject. One non-limiting example of such a dressing 1028 is Tegaderm™, a transparent medical dressing available from 3M Corporation. A lower housing 1016 includes a generally rectangular shaped opening 1030 that aligns with another opening 1032 in the dressing 1028.

Referring to FIG. 13, another “bottom up” exploded view of the ultrasound patch 1010 illustrates the location of ultrasonic transducers and integrated CMOS chip (generally indicated by 1034) on the circuit board 1018. An acoustic lens 1036 mounted over the transducers/CMOS chip 1034 is configured to protrude through openings 1030 and 1032 to make contact with the skin of a subject. In some embodiments, the ultrasonic transducers may be arranged in a two-dimensional array. In some embodiments, the ultrasonic transducers may be arranged in a 1.75-dimensional array (as described further below).

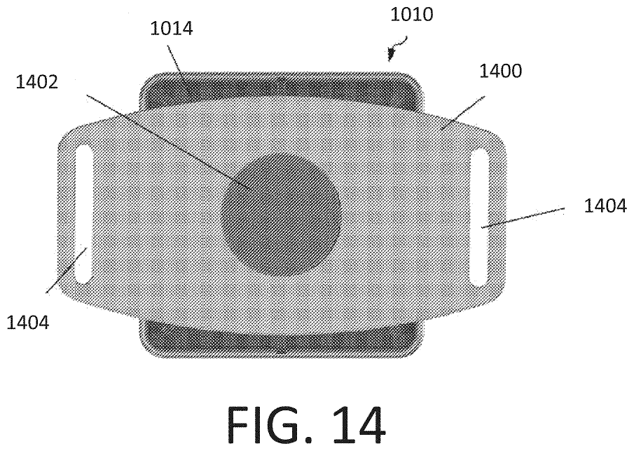

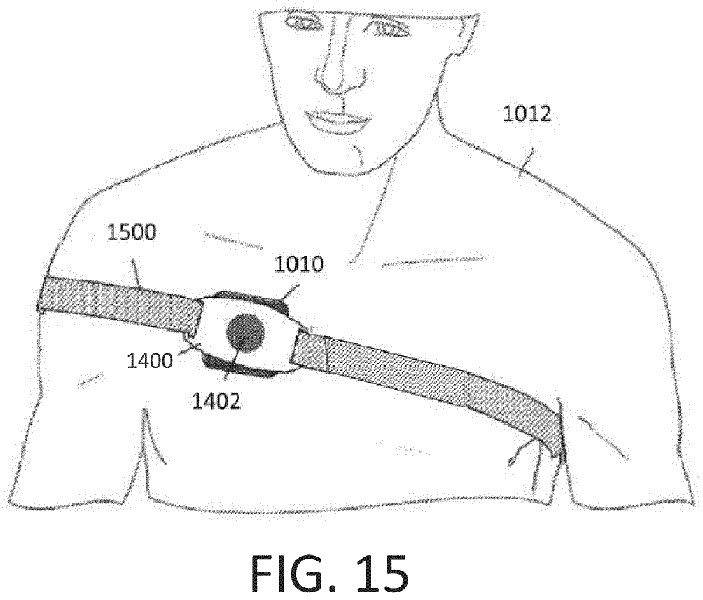

Referring to FIG. 14, an alternative fastening mechanism for the ultrasound patch 1010 in accordance with certain embodiments described herein is illustrated. In the embodiment shown, the ultrasound patch 1010 further includes a buckle 1400 affixed to the upper housing 1014 via a post 1402 using, for example, a threaded engagement between the buckle 1400 and the post 1402. Other attachment configurations are also contemplated, however. As further shown in FIG. 14, the buckle 1400 includes a pair of slots 1404 that in turn accommodate a strap 1500 (FIG. 15).

FIG. 15 shows an example of the ultrasound patch 1010 fastened to the subject 1012 using the strap 1500 in accordance with certain embodiments described herein. In this example, the strap 1500 is wrapped around the subject 1012 and appropriately tightened in order to secure the ultrasound patch 1010 to a desired location on the subject 1012 for acquisition of desired ultrasound data and/or delivery of desired ultrasound energy.

In some embodiments, the ultrasound patch 1010 may weigh no more than 2 kg (e.g., no more than 1 kg). In some embodiments, the volume of the wearable ultrasound device may be no greater than 250 cm³ (e.g., no greater than 125 cm³, or no greater than 50 cm³). In some embodiments, the ultrasound transducers of the ultrasound patch 1010 may be arranged in an array, and the height of the wearable ultrasound device along the direction orthogonal to the array of ultrasound transducers (i.e., orthogonal to the face of the array) may be no greater than 7 cm (e.g., no greater than 5 cm.) In some embodiments, the height of the wearable ultrasound device along the direction orthogonal to the array of ultrasound transducers may be no greater than a dimension of the array of ultrasound transducers (i.e., the length or width of the array). As described above, the portability/wearability (i.e., the acceptably small size/weight) of the ultrasound patch 1010 may be due, in part, to monolithically integrating ultrasound transducers onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application).

Additional information regarding the fabrication and integration of ultrasound transducers with CMOS wafers (e.g., to form the CMOS chip 1034, which may be an ultrasound-on-a-chip) may be found in U.S. Pat. No. 9,067,779 titled MICROFABRICATED ULTRASONIC TRANSDUCERS AND RELATED APPARATUS AND METHODS, granted on Jun. 30, 2015 (and assigned to the assignee of the present application), the contents of which are incorporated by reference herein in their entirety. Additional information regarding the circuit components of the CMOS chip 1034 may be found in U.S. Pat. No. 9,521,991 titled “MONOLITHIC ULTRASONIC IMAGING DEVICES, SYSTEMS, AND METHODS,” granted on Dec. 20, 2016 (and assigned to the assignee of the instant application), the contents of which are incorporated by reference herein in their entirety.

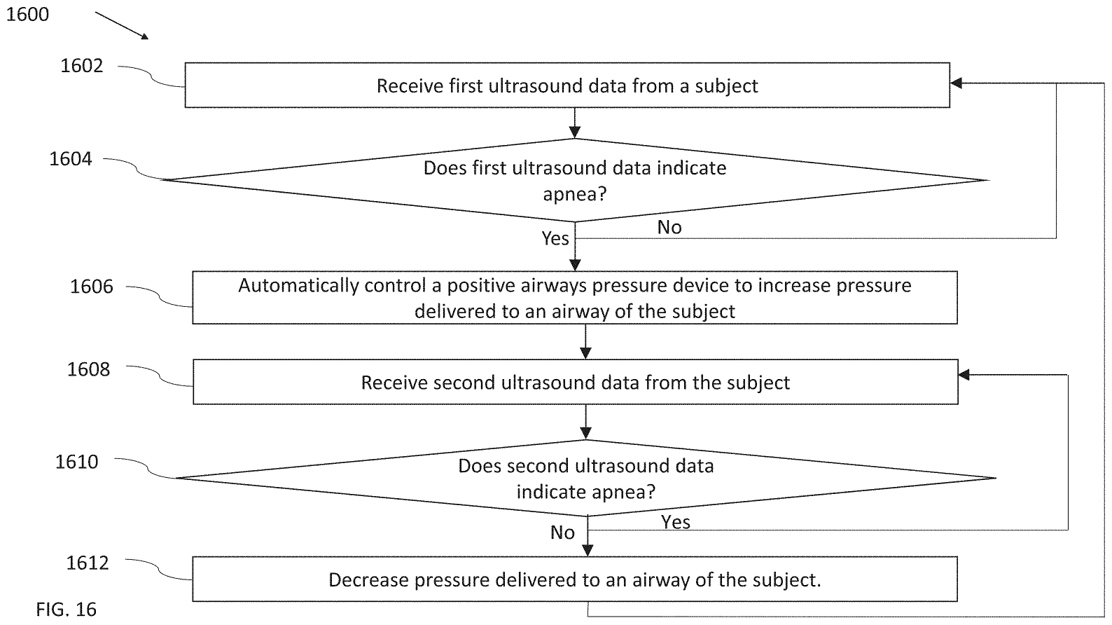

FIG. 16 shows an example process 1600 for delivering pressure based on ultrasound data, in accordance with certain embodiments described herein. The process 1600 may be performed by, for example, a processing device (e.g., processing device 108), a wearable ultrasound device (e.g., ultrasound device 104), an adapter (e.g., adapter 220), a valve (e.g., valve 356), or a positive airway pressure device (e.g., positive airway pressure device 106). In particular, processing circuitry executing instructions stored in memory circuitry in the device may perform the process 1600. Further description of the acts of process 1600 may be found above with reference to FIGS. 1-9.

In act 1602, the processing circuitry receives first ultrasound data from a subject. The process 1600 proceeds to act 1604.

In act 1604, the processing circuitry automatically determines whether the first ultrasound data indicates apnea. If the processing circuitry determines that the first ultrasound data indicates apnea, the process 1600 proceeds from act 1604 to act 1606. If the processing circuitry determines that the ultrasound data does not indicate apnea, the process 1600 repeats act 1602.

In act 1606, the processing circuitry automatically controls a positive airway pressure device coupled to the subject to increase pressure delivered to a subject's airway. The process 1600 proceeds from act 1606 to act 1608.

In act 1608, the processing circuitry receives second ultrasound data (which may be different than the first ultrasound data). The process 1600 proceeds from act 1608 to act 1610.

In act 1610, the processing circuitry automatically determines whether the second ultrasound data indicates apnea. If the processing circuitry determines that the second ultrasound data does not indicate apnea, the process 1600 proceeds from act 1610 to act 1612. If the processing circuitry determines that the ultrasound data indicates apnea, the process 1600 repeats act 1608.

In act 1612, the processing circuitry automatically controls the positive airway pressure device to decrease pressure delivered the subject's airway. The process 1600 proceeds from act 1612 to act 1602.

In some embodiments, the process 1600 may lack acts 1602, 1604, and 1606. In some embodiments, the process 1600 may lack acts 1608, 1610, and 1612. In some embodiments, at act 1604 the processing circuitry may determine whether the first ultrasound data does not indicate apnea, at act 1606 the processing circuitry may automatically control the positive airway pressure device to decrease the pressure delivered to the subject's airway, at act 1610 the processing circuitry may determine whether the second ultrasound data indicates apnea, and at act 1612 the processing circuitry may automatically control the positive airway pressure device to increase the pressure delivered to the subject's airway.

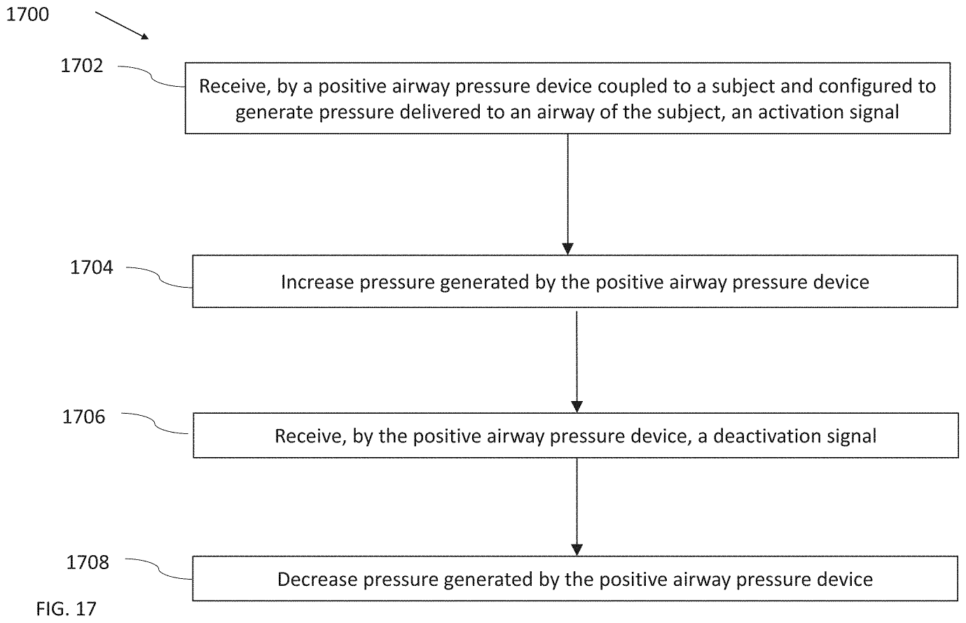

FIG. 17 shows an example process 1700 for delivering pressure to a subject, in accordance with certain embodiments described herein. The process 1700 is performed by a positive airway pressure device (e.g., positive airway pressure device 106) coupled to a subject and configured to generate pressure delivered to an airway of the subject. In particular, processing circuitry (e.g., processing circuitry 438) executing instructions stored in memory circuitry (e.g., memory circuitry 440) in the positive airway pressure device may perform the process 1700. Further description of the acts of process 1700 may be found above with reference to FIGS. 1-9.

In act 1702, the positive airway pressure device receives an activation signal. The activation signal may be received, for example, from a processing device (e.g., processing device 108) in communication with a wearable ultrasound device (e.g., ultrasound device 104) coupled to a subject, or from the wearable ultrasound device itself. The process 1700 proceeds from act 1702 to act 1704.

In act 1704, based on receiving the activation signal in act 1702, the positive airway pressure device increases pressure generated by the positive airway pressure device. The process 1700 proceeds from act 1704 to act 1706.

In act 1706, the positive airway pressure device receives a deactivation signal. The deactivation signal may be received, for example, from the processing device or from the wearable ultrasound device. The process 1700 proceeds from act 1706 to act 1708.

In act 1708, based on receiving the deactivation signal in act 1706, the positive airway pressure device decreases pressure generated by the positive airway pressure device.

In some embodiments, the process 1700 may lack acts 1702 and 1704. In some embodiments, the process 1700 may lack acts 1706 and 1708. In some embodiments, at act 1702 the positive airway pressure device may receive the deactivation signal, at act 1704 the positive airway pressure device may decrease pressure generated by the positive airway pressure device, at act 1706 the positive airway pressure device may receive the activation signal, and at act 1708 the positive airway pressure device may increase pressure generated by the positive airway pressure device.

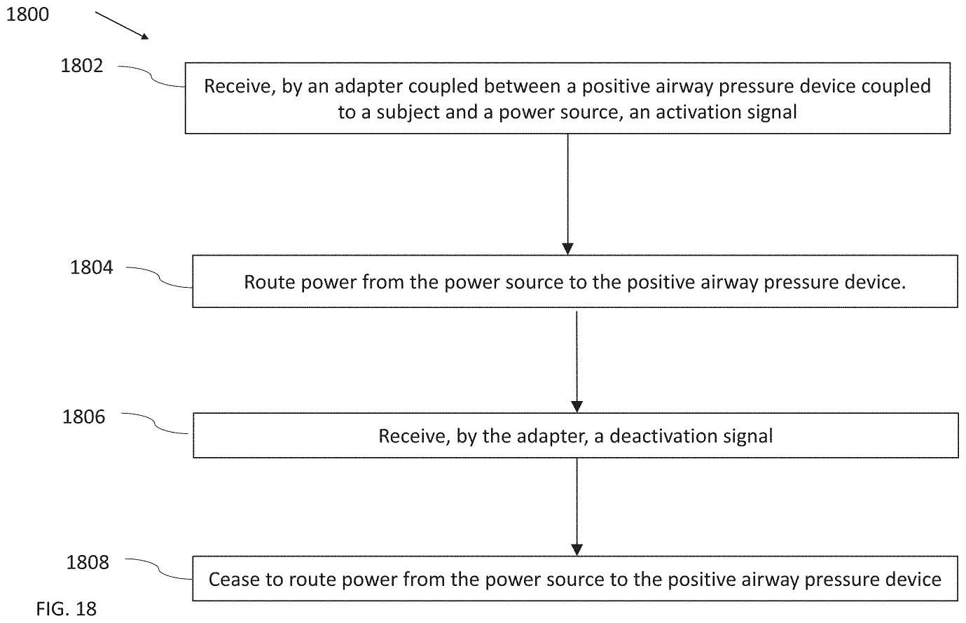

FIG. 18 shows an example process 1800 for delivering pressure to a subject, in accordance with certain embodiments described herein. The process 1800 is performed by an adapter (e.g., adapter 220) coupled between a positive airway pressure device (e.g., positive airway pressure device 106) coupled to a subject and a power source (e.g., power source 116). In particular, processing circuitry (e.g., processing circuitry 644) executing instructions stored in memory circuitry (e.g., memory circuitry 646) in the adapter may perform the process 1800. Further description of the acts of process 1800 may be found above with reference to FIGS. 1-9.

In act 1802, the adapter receives an activation signal. The activation signal may be received, for example, from a processing device (e.g., processing device 108) in communication with a wearable ultrasound device (e.g., ultrasound device 104) coupled to a subject, or from the wearable ultrasound device itself. The process 1800 proceeds from act 1802 to act 1804.

In act 1804, based on receiving the activation signal in act 1802, the adapter routes power from the power source to the positive airway pressure device. The process 1800 proceeds from act 1804 to act 1806.

In act 1806, the adapter receives a deactivation signal. The deactivation signal may be received, for example, from the processing device or from the wearable ultrasound device itself. The process 1800 proceeds from act 1806 to act 1808.

In act 1808, based on receiving the deactivation signal in act 1806, the adapter ceases to route power from the power source to the positive airway pressure device.

In some embodiments, the process 1800 may lack acts 1802 and 1804. In some embodiments, the process 1800 may lack acts 1806 and 1808. In some embodiments, at act 1802 the adapter may receive the deactivation signal, at act 1804 the adapter may cease to route power from the power source to the positive airway pressure device, at act 1806 the adapter may receive the activation signal, and at act 1808 the adapter may route power from the power source to the positive airway pressure device.

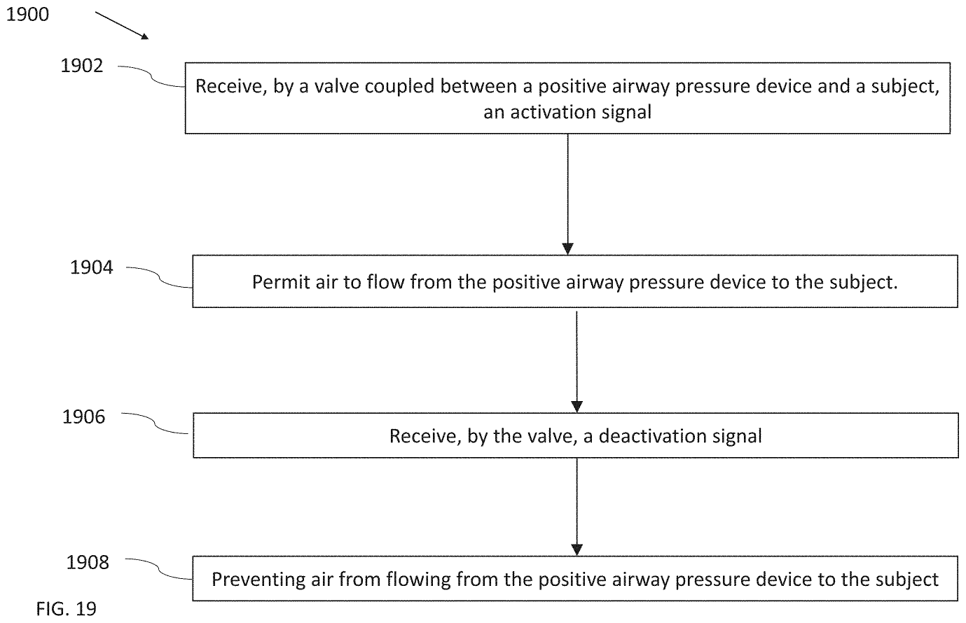

FIG. 19 shows an example process 1900 for delivering pressure to a subject, in accordance with certain embodiments described herein. The process 1800 is performed by a valve (e.g., valve 356) coupled between a positive airway pressure device (e.g., positive airway pressure device 106) and a subject. In particular, processing circuitry (e.g., processing circuitry 858) executing instructions stored in memory circuitry (e.g., memory circuitry 860) in the valve may perform the process 1900. Further description of the acts of process 1900 may be found above with reference to FIGS. 1-9.

In act 1902, the valve receives an activation signal. The activation signal may be received, for example, from a processing device (e.g., processing device 108) in communication with a wearable ultrasound device (e.g., ultrasound device 104) coupled to a subject, or from the wearable ultrasound device itself. The process 1900 proceeds from act 1902 to act 1904.

In act 1904, based on receiving the activation signal in act 1902, the valve permits air to flow from the positive airway pressure device to the subject. The process 1900 proceeds from act 1904 to act 1906.

In act 1906, the valve receives a deactivation signal. The deactivation signal may be received, for example, from the processing device or from the wearable ultrasound device itself. The process 1900 proceeds from act 1906 to act 1908.

In act 1908, based on receiving the deactivation signal in act 1906, the valve prevents air from flowing from the positive airway pressure device to the subject.

In some embodiments, the process 1900 may lack acts 1902 and 1904. In some embodiments, the process 1900 may lack acts 1906 and 1908. In some embodiments, at act 1902 the valve may receive the deactivation signal, at act 1904 the valve may prevent air from flowing from the positive airway pressure device to the subject, at act 1906 the valve may receive the activation signal, and at act 1908 the valve may permit air to flow from the positive airway pressure device to the subject.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

In some embodiments, calibration of the systems described herein may be performed for a particular subject. For example, ultrasound data may be collected while the subject is breathing and not breathing to characterize the ultrasound data in these two conditions. For example, in the case of determining apnea based on lung sliding, characterization may include measuring speed, distance, direction, and time of lung sliding during breathing and not breathing. A speed, distance, direction, and/or time of lung sliding above a certain threshold may be considered to be indicative of lack of apnea, while below a certain threshold may be considered to be indicative of apnea. In the case of determining apnea based on movement of internal abdominal organs, characterization may include measuring speed, distance, direction, and time of movement of internal abdominal organs. A speed, distance, direction, and/or time of movement of internal abdominal organs above a certain threshold may be considered to be indicative of lack of apnea, while below a certain threshold may be considered to be indicative of apnea. As another example, training data from a particular subject, including ultrasound data and labels indicating whether the ultrasound data was collected from the subject during breathing or not breathing, may be used to train a deep learning network to determine whether given ultrasound data indicates apnea or not. During collection of data while the subject is breathing or not breathing, the subject may be explicitly instructed to breathe and not breathe at specific times, or other data collected from the subject, such as respiratory airflow, may be used to determine whether the subject is breathing or not.

Aspects of the technology described herein relate to the application of automated image processing techniques to analyze images, such as ultrasound images. In some embodiments, the automated image processing techniques may include machine learning techniques such as deep learning techniques. Machine learning techniques may include techniques that seek to identify patterns in a set of data points and use the identified patterns to make predictions for new data points. These machine learning techniques may involve training (and/or building) a model using a training data set to make such predictions. The trained model may be used as, for example, a classifier that is configured to receive a data point as an input and provide an indication of a class to which the data point likely belongs as an output.

Deep learning techniques may include those machine learning techniques that employ neural networks to make predictions. Neural networks typically include a collection of neural units (referred to as neurons) that each may be configured to receive one or more inputs and provide an output that is a function of the input. For example, the neuron may sum the inputs and apply a transfer function (sometimes referred to as an “activation function”) to the summed inputs to generate the output. The neuron may apply a weight to each input, for example, to weight some inputs higher than others. Example transfer functions that may be employed include step functions, piecewise linear functions, and sigmoid functions. These neurons may be organized into a plurality of sequential layers that each include one or more neurons. The plurality of sequential layers may include an input layer that receives the input data for the neural network, an output layer that provides the output data for the neural network, and one or more hidden layers connected between the input and output layers. Each neuron in a hidden layer may receive inputs from one or more neurons in a previous layer (such as the input layer) and provide an output to one or more neurons in a subsequent layer (such as an output layer).

A neural network may be trained using, for example, labeled training data. The labeled training data may include a set of example inputs and an answer associated with each input. For example, the training data may include a plurality of ultrasound images that are each labeled with an anatomical feature (e.g., an indication of apnea) that is contained in the respective ultrasound image. In this example, the ultrasound images may be provided to the neural network to obtain outputs that may be compared with the labels associated with each of the ultrasound images. One or more characteristics of the neural network (such as the interconnections between neurons (referred to as edges) in different layers and/or the weights associated with the edges) may be adjusted until the neural network correctly classifies most (or all) of the input images.

Once the training data has been created, the training data may be loaded to a database (e.g., an image database) and used to train a neural network using deep learning techniques. Once the neural network has been trained, the trained neural network may be deployed to one or more computing devices. It should be appreciated that the neural network may be trained with any number of sample subject images, although it will be appreciated that the more sample images used, the more robust the trained model data may be.

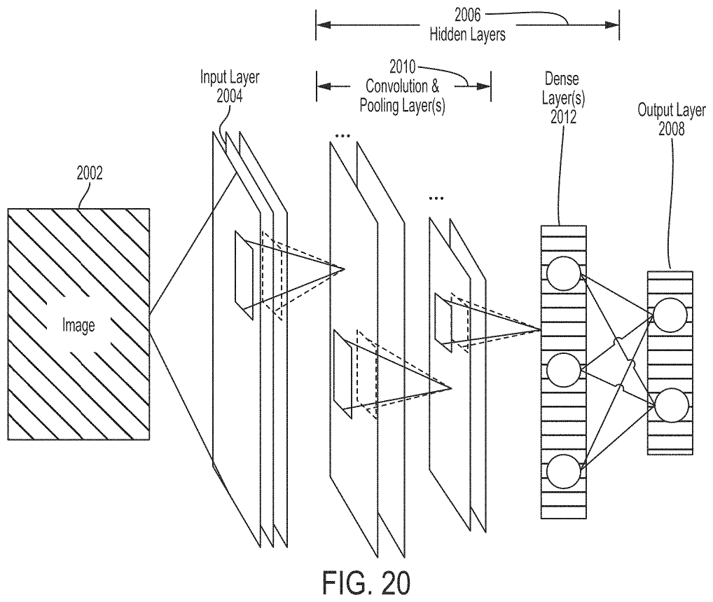

In some applications, a neural network may be implemented using one or more convolution layers to form a convolutional neural network. An example convolutional neural network is shown in FIG. 20 that is configured to analyze an image 2002. As shown, the convolutional neural network includes an input layer 2004 to receive the image 2002, an output layer 2008 to provide the output, and a plurality of hidden layers 2006 connected between the input layer 2004 and the output layer 2008. The plurality of hidden layers 2006 includes convolution and pooling layers 2010 and dense layers 2012.

The input layer 2004 may receive the input to the convolutional neural network. As shown in FIG. 20, the input the convolutional neural network may be the image 2002. The image 2002 may be, for example, an ultrasound image.

The input layer 2004 may be followed by one or more convolution and pooling layers 2010. A convolutional layer may include a set of filters that are spatially smaller (e.g., have a smaller width and/or height) than the input to the convolutional layer (e.g., the image 2002). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 2010 may be followed by dense layers 2012. The dense layers 2012 may include one or more layers each with one or more neurons that receives an input from a previous layer (e.g., a convolutional or pooling layer) and provides an output to a subsequent layer (e.g., the output layer 2008). The dense layers 2012 may be described as “dense” because each of the neurons in a given layer may receive an input from each neuron in a previous layer and provide an output to each neuron in a subsequent layer. The dense layers 2012 may be followed by an output layer 2008 that provides the output of the convolutional neural network. The output may be, for example, an indication of which class, from a set of classes, the image 2002 (or any portion of the image 2002) belongs to.

It should be appreciated that the convolutional neural network shown in FIG. 20 is only one example implementation and that other implementations may be employed. For example, one or more layers may be added to or removed from the convolutional neural network shown in FIG. 20. Additional example layers that may be added to the convolutional neural network include: a rectified linear units (ReLU) layer, a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

Convolutional neural networks may be employed to perform any of a variety of functions described herein. For example, a convolutional neural network may be employed to identify an anatomical feature (e.g., an indication of apnea) in an ultrasound image. For further discussion of deep learning techniques, see U.S. patent application Ser. No. 15/626,423 titled “AUTOMATIC IMAGE ACQUISITION FOR ASSISTING A USER TO OPERATE AN ULTRASOUND DEVICE,” filed on Jun. 19, 2017 (and assigned to the assignee of the instant application), which is incorporated by reference herein in its entirety.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “substantially”, “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
processing circuitry configured to:
receive first ultrasound data collected from a subject by a wearable ultrasound device;
automatically determine that the first ultrasound data indicates apnea; and
responsive to a determination that the first ultrasound data indicates apnea, automatically control a positive airway pressure device coupled to the subject to increase pressure delivered to an airway of the subject.

2. The apparatus of claim 1, wherein the processing circuitry is configured, when automatically determining that the first ultrasound data indicates apnea, to automatically determine that the subject is experiencing apnea.

3. The apparatus of claim 1, wherein the processing circuitry is configured, when automatically determining that the first ultrasound data indicates apnea, to input the first ultrasound data to a statistical model trained to determine whether inputted ultrasound data indicates apnea.

4. The apparatus of claim 1, wherein the processing circuitry is configured, when automatically determining that the first ultrasound data indicates apnea, to determine that the first ultrasound data indicates an absence of lung sliding.

5. The apparatus of claim 1, wherein the processing circuitry is configured, when automatically determining that the first ultrasound data indicates apnea, to determine that the first ultrasound data indicates an absence of movement of internal abdominal organs.

6. The apparatus of claim 1, wherein the processing circuitry is further configured to:
receive second ultrasound data collected from the subject by the wearable ultrasound device;
automatically determine that the second ultrasound data does not indicate apnea; and
responsive to a determination by the processing circuitry that second ultrasound data does not indicate apnea, automatically control the positive airway pressure device to decrease pressure delivered to the airway of the subject.

7. The apparatus of claim 6, wherein the processing circuitry is configured, when automatically determining that the second ultrasound data does not indicate apnea, to automatically determine that the subject is not experiencing apnea.

8. The apparatus of claim 6, wherein the processing circuitry is configured, when determining that the second ultrasound data does not indicate apnea, to input the first ultrasound data to a statistical model trained to determine whether inputted ultrasound data does not indicate apnea.

9. The apparatus of claim 6, wherein the processing circuitry is configured, when automatically determining that the second ultrasound data does not indicate apnea, to determine that the second ultrasound data indicates lung sliding.

10. The apparatus of claim 6, wherein the processing circuitry is configured, when automatically determining that the second ultrasound data does not indicate apnea, to determine that the second ultrasound data indicates movement of internal abdominal organs.

11. The apparatus of claim 1, wherein:
the positive airway pressure device comprises the processing circuitry; and
the processing circuitry is configured, when automatically controlling the positive airway pressure device to increase the pressure delivered to the airway of the subject, to increase pressure generated by the positive airway pressure device.

12. The apparatus of claim 6, wherein:
the positive airway pressure device comprises the processing circuitry; and
the processing circuitry is configured, when automatically controlling the positive airway pressure device to decrease the pressure delivered to the airway of the subject, to decrease pressure generated by the positive airway pressure device.

13. The apparatus of claim 1, wherein:
an adapter coupled between the positive airway pressure device and a power source comprises the processing circuitry; and
the processing circuitry is configured, when automatically controlling the positive airway pressure device to increase the pressure delivered to the airway of the subject, to route power from the power source to the positive airway pressure device.

14. The apparatus of claim 6, wherein:
an adapter coupled between the positive airway pressure device and a power source comprises the processing circuitry; and
the processing circuitry is configured, when automatically controlling the positive airway pressure device to decrease the pressure delivered to the airway of the subject, to cease to route power from the power source to the positive airway pressure device.

15. The apparatus of claim 1, wherein:
a valve coupled between the positive airway pressure device and the subject comprises the processing circuitry; and
the processing circuitry is configured, when automatically controlling the positive airway pressure device to increase the pressure delivered to the airway of the subject, to permit air to flow from the positive airway pressure device to the airway of the subject.

16. The apparatus of claim 6, wherein:
a valve coupled between the positive airway pressure device and the subject comprises the processing circuitry; and
the processing circuitry is configured, when automatically controlling the positive airway pressure device to decrease the pressure delivered to the airway of the subject, to prevent air to flow from the positive airway pressure device to the airway of the subject.

17. The apparatus of claim 1, wherein the processing circuitry is configured, when automatically controlling the positive airway pressure device to increase the pressure delivered to the airway of the subject, to generate an activation signal configured to trigger the positive airway device to increase the pressure generated by the positive airway pressure device.

18. The apparatus of claim 6, wherein the processing circuitry is configured, when automatically controlling the positive airway pressure device to increase the pressure delivered to the airway of the subject, to generate a deactivation signal configured to trigger the positive airway device to decrease the pressure generated by the positive airway pressure device.

19. The apparatus of claim 1, wherein the processing circuitry is configured, when automatically controlling the positive airway pressure device to increase the pressure delivered to the airway of the subject, to generate an activation signal configured to trigger an adapter coupled between the positive airway pressure device and a power source to route the power from the power source to the positive airway pressure device.

20. The apparatus of claim 6, wherein the processing circuitry is configured, when automatically controlling the positive airway pressure device to increase the pressure delivered to the airway of the subject, to generate a deactivation signal configured to trigger an adapter coupled between the positive airway pressure device and a power source to cease to route the power from the power source to the positive airway pressure device.

21. The apparatus of claim 1, wherein the processing circuitry is configured, when automatically controlling the positive airway pressure device to increase the pressure delivered to the airway of the subject, to generate an activation signal configured to trigger a valve coupled between the positive airway pressure device and the subject to permit the air to flow from the positive airway pressure device to the airway of the subject.

22. The apparatus of claim 6, wherein the processing circuitry is configured, when automatically controlling the positive airway pressure device to increase the pressure delivered to the airway of the subject, to generate a deactivation signal configured to trigger a valve coupled between the positive airway pressure device and the subject to prevent the air to flow from the positive airway pressure device to the airway of the subject.

23. The apparatus of claim 17, wherein the wearable ultrasound device comprises the processing circuitry.

24. The apparatus of claim 1, wherein the wearable ultrasound device comprises a patch configured to couple to the subject's skin.

25. The apparatus of claim 17, wherein a processing device in communication with the wearable ultrasound device comprises the processing circuitry.
